(19) United States
(12) Patent Application Publication
(10) Pub. No.: US 2025/0315952 A1
(43) Pub. Date: Oct. 9, 2025

(54) ARTIFICIAL INTELLIGENCE-BASED TOOL FOR MYOCARDIAL BLOOD FLOW PARAMETRIC MAPPING TO DIAGNOSE CORONARY ARTERY DISEASE WITH 82RB POSITRON EMISSION TOMOGRAPHY

(71) Applicant: Jubilant Draximage Inc., Montreal (CA)

(72) Inventors: Eric James Moulton, Montreal (CA); Robert A. DeKemp, Ottawa (CA); Chad Roger Ronald Nicholas Hunter, Ottawa (CA)

(21) Appl. No.: 19/245,299

(22) Filed: Jun. 21, 2025

Related U.S. Application Data
(63) Continuation-in-part of application No. 18/412,857, filed on Jan. 15, 2024.
Application No. 18/412,857 is a continuation-in-part of application No. 18/242,131, filed on Sep. 5, 2023, now Pat. No. 12,268,547.
(60) Provisional application No. 63/374,732, filed on Sep. 6, 2022.
(60) Provisional application No. 63/499,399, filed on May 1, 2023.

(51) Int. Cl. G06T 7/00; A61B 5/00; A61B 5/026; A61B 6/00; A61B 6/03; A61B 6/50; G06T 7/62

(52) U.S. Cl. CPC G06T 7/0012; A61B 5/0035; A61B 5/0263; A61B 5/7264; A61B 5/7275; A61B 6/037; A61B 6/504; A61B 6/5247; A61B 6/541; G06T 7/62; G06T 2207/30104

(57) ABSTRACT

The present invention discloses methods for automatically computing an arterial input function from one or more regions of interest, the method comprising: a. obtaining a plurality of dynamic image data sets comprising volumetric image data from the regions of interest over multiple scanning intervals; b. utilizing an artificial neural network to segment the plurality of dynamic image data sets displaying one or more arterial input function(s) (AIF) in the region(s) of interest; c. automatically estimating, using artificial intelligence, an arterial input function based on plurality of dynamic image data sets combined with one or more time activity curves (TAC) in the region(s) of interest in target organ(s); and d. computing a pre-trained predictive pharmacokinetic AI model arterial input function using time activity curve input associated with region(s) of interest of target organ(s).

### CROSS-REFERENCE TO RELATED APPLICATIONS

The application is a continuation-in-part of application Ser. No. 18/412,857, which is a continuation-in-part of application No. 18/242, 131, filed on Sep. 5, 2023, now Pat. No. 12,268,547, which claims the benefit of provisional application No. 63/499,399, filed on May 1, 2023, and provisional application No. 63/374,732, filed on Sep. 6, 2022.

### BACKGROUND OF THE INVENTION

Nuclear medicine employs radioactive material for therapy and diagnostic imaging. Different types of diagnostic imaging are available that utilize doses of radiopharmaceuticals. The desired doses of radiopharmaceuticals can be injected or infused into a patient prior to or during the diagnostic imaging procedure, wherein the radiopharmaceuticals can be absorbed by the cells or adhered to the cells of a target organ of the patient and emit radiation. The scanner or detector of the diagnostic imaging process can then detect the emitted radiation in order to generate an image of an organ. For example, to image body tissue such as the heart muscle, a patient can be infused or injected with ⁸²Rb (i.e., Rubidium-82). The diagnostic imaging procedure can detect the radiation of ⁸²Rb and facilitate better images of myocardium to diagnose any clinical disease.

Radioisotopes play a pivotal role in diagnosis and mitigation of various disease conditions. For example, ⁶⁰Co in treatment of cancer, ¹³¹I in treatment of hyperthyroidism, ¹⁴C in breath tests, ^{99m}Tc and ⁸²Rb as tracers in myocardial perfusion imaging.

Further, ⁸²Rb is produced in-situ by radioactive decay of ⁸²Sr (Strontium-82). Rubidium elution systems utilize doses of ⁸²Rb generated by elution within a radioisotope generator, and infuse or inject the radioactive solution into a patient.

Previously, preclinical studies in dogs showed that myocardial uptake of ⁸²Rb radionuclide was directly related to myocardial blood flow (MBF). Dynamic cine myocardial perfusion imaging (MPI) with radioisotopes can produce accurate prediction of myocardial blood flow (MBF) and myocardial flow reserve (MFR) through pharmacokinetic modeling of various compartment models, such as the one tissue compartment model. Typically, MBF estimation begins with segmentation of the left ventricle (LV) myocardium followed by tracer kinetic modeling in a limited number of two-dimensional (2D) polar-map sectors or segments. However, two-dimensional (2D) polar-map segments have drawbacks. Few cardiac disorders related to flow in small regional flow defect can be visualized properly in a two-dimensional (2D) polar-map. Therefore, there is an alternate method of myocardial perfusion imaging by visualizing three-dimensional (3D) parametric maps of MBF. But there are some disadvantages in practice of visualizing three-dimensional (3D) parametric maps of MBF. Further, in identifying small regional flow defects are not always visible. Additionally, generating the three-dimensional (3D) parametric maps is time consuming and is less stable, particularly in areas of high arterial blood concentration where partial volume correction is notoriously difficult. These disadvantages discourage the healthcare providers to adopt the use of 3D parametric maps of MBF for estimating myocardial blood flow. Therefore, there is a need for an alternative approach to generate more stable three-dimensional (3D) parametric maps in lesser time to estimate not only the myocardial blood blow and flow reserve for cardiac PET but for any pharmacokinetic modeling for other organs, such as the brain, kidneys, lower extremities, etc.

Previously developed methods for 3D MBF parametric mapping have generally been validated from analytic point-of-view by correlating the MBF values obtained with reference 2D polar map processing and 3D parametric maps for the whole left ventricle (LV) and the three vessel territories (LAD, RCA, and LCx). However, no study has yet to verify that parametric map-derived MBF values for ⁸²Rb PET correlate with the presence of coronary artery disease (CAD). The inventors of the present invention validated an AI model training paradigm to perform MBF parametric mapping by evaluating its diagnostic ability to predict CAD and compared its performance with traditional polar map kinetic modelling.

⁸²Rb PET imaging was performed using a single fixed dose for all patients, due in part to limitations of early-generation tracer delivery systems. The undesirable effect of old PET imaging systems can be mitigated to some extent by using the advanced and latest generation ⁸²Rb elution system. The present inventors observed that by using the 3D parametric imaging of myocardial perfusion with ⁸²Rb PET, they can accurately estimate and/or predict myocardial blood flow (MBF) and/or myocardial flow reserve (MFR).

Myocardial automated estimation generally includes image preprocessing, arterial input function (AIF) selection, deconvolution computation, parametric map generation, blood volume computation, and the like, wherein AIF participates in deconvolution operation. In conventional kinetic modelling of the one tissue compartment model (1TCM) using non-linear least squares regression for estimating MBF, the radio tracer residual curve can be obtained by deconvolution operation of the time activity curve (TAC) and AIF, and the above-mentioned various hemodynamic parameters and parameter diagrams thereof can be obtained by further calculation of the TAC.

The AIF points typically select arterial voxels located on the left ventricle for myocardium and these curves typically have a peak height, small peak width, early peak time profile. At present, the AIF selection method mainly comprises the methods of manual selection by the operator or technician and constructing a curve characteristic weighting model. However, the manual selection method has the disadvantages of long time consumption, low repeatability and reliance on operator experience; the method for constructing the curve characteristic weighting model needs to manually construct characteristics and design a complex mathematical model.

Further, for accurate pharmacokinetic modeling, for example with a one-tissue compartment model (1TCM), one relies on proper placement of a region of interest (ROI) for deriving an arterial input function (AIF). However, inter-operator variability is a major source of difference in kinetic modeling parameter estimates. Therefore, previous work has taken advantage of artificial intelligence (AI) mostly in a supervised fashion to automatically reproduce ROIs or AIFs from expert annotations. However, for certain applications, the optimal placement of the ROI is still a subject of research. In the case of myocardial blood flow (MBF) estimation, the ROI is typically placed in the left ventricle (LV) although previous research has suggested that other areas, such as the left atrium (LA), could yield more reproducible MBF measures. In the present invention, the inventors used a purely self-supervised approach with an AI algorithm that generates a ROI and thus, AIF optimized to minimize the fits of a specific pharmacokinetic model. Such a data-driven approach cannot only potentially improve estimates of kinetic modeling parameters by incorporating the appropriate physical equations into its training, such as those for MBF, but also validate existing methods of ROI placement.

Interestingly, parametric mapping of MBF simultaneously yields 3D parametric maps of fractional or total blood volume (TBV—given as a fraction of the pure arterial blood contribution to the TAC). 3D TBV Parametric maps highlight blood pools where arterial blood is most prominent, and provide a means to directly view volumes of the various blood pools, including the left and right ventricle. Traditionally, Positron Emission Tomography (PET) myocardial perfusion imaging (MPI) using Rubidium-82 (⁸²Rb) has served as a robust modality for assessing left ventricular (LV) perfusion and function. However, its utility in evaluating right ventricular (RV) function has been limited. This limitation arises primarily due to inherently poor tracer uptake in the RV myocardium and suboptimal delineation of the blood pool during gated imaging acquisition.

Conventional gated uptake MPI methods often fail to provide accurate biventricular volumetric and functional parameters, particularly for the RV. Cardiac Magnetic Resonance (CMR) imaging remains the gold standard for such assessments, but it is not always accessible or feasible in clinical settings. Therefore, there exists a need for a method that enables accurate, non-invasive, and widely accessible estimation of biventricular function using existing PET imaging infrastructure. TBV parametric mapping from the same invention can be used to assess LV and RV volumes and ejection fractions (EF) as a more accurate means than conventional gated uptake MPI where the LV and RV blood pools are indirectly viewed.

### SUMMARY OF THE INVENTION

The present invention aims to provide an image processing method to assess quantitative myocardial blood flow (MBF) and/or myocardial flow reserve (MFR).

The present invention discloses methods for obtaining the arterial input function (AIF) by using a fully automated unsupervised trained neural network model, to improve accuracy and the robustness of calculation and analysis of 3D parametric map.

The object of the present invention is to provide an alternate method to use 3D voxel-wise parametric imaging data to estimate and/or predict quantitative myocardial blood flow (MBF) and/or myocardial flow reserve (MFR), which may better highlight small regional flow defects. More precisely, the inventors of the present invention estimate the myocardial blood flow (MBF) and/or myocardial flow reserve (MFR), wherein the image series fit to a one-tissue-compartment model yielding voxel-wise parametric maps comparing the projected data onto a two-dimensional (2D) polar map of the left ventricle (LV). The present invention, therefore, has an advantage of producing regional flow and reserve values, which may highlight better the small regional flow defects and are independent of LV polar-map segmentation.

The object of the present invention is to provide an image processing method to assess quantitative myocardial blood flow and/or myocardial flow reserve, wherein the image reconstruction algorithms are designed to improve the quality of images by using the AI algorithm to enhance the image reconstruction quality, which is intended to do the image processing faster and reduce the doses of nuclear medicine up to 10 times during the myocardial perfusion imaging (MPI).

The object of the present invention for AI models can generate blood flow parametric maps with high accuracy and in a timeframe acceptable for clinical use and thus may enable future clinical implementation.

The object of the present invention is to provide an image processing method to assess quantitative myocardial blood flow and/or myocardial flow reserve, wherein 3D parametric images of MBF generated by present invention also recommend the calcium scoring.

In an embodiment of the present invention, an image processing method to assess quantitative myocardial blood flow and myocardial flow reserve, comprises the steps of:
a. a step of pre-processing of images comprising:
   (i) reconstructing dynamic cine 3D tomographic myocardial perfusion imaging (MPI) data,
   (ii) isolating value at voxel (i, j, k) for each time point t; where i is from 1 to N,
   (iii) optionally, denoising to improve the quality of image,
   (iv) extracting blood input function from a region of interest (ROI) of the left ventricle blood cavity or other arterial blood region of region of interest (ROI),
   (v) estimating the distribution volume (DV), given by the ratio of uptake and washout rates (K1/K2) to stabilize and improve estimation of K1 and total blood volume (TBV) and subsequent myocardial blood flow measures, and
   (vi) data normalization by dividing by the maximum of the blood input function;
b. a step of assessing the individual signals pre-processed in step (a) in order to generate K1 and TBV parametric maps from the one tissue compartment model using an artificial neural network;
c. a step of post-processing of K1 and TBV parametric maps; and of rest and stress myocardial blood flow to estimate myocardial flow reserve (MFR) and/or coronary flow reserve (CFR).

An embodiment of the present invention includes the image processing method to assess quantitative myocardial blood flow and myocardial flow reserve, wherein the image reconstruction of arrays is a dynamic cine series comprising the 3D tomographic voxel (i, j, k) from PET reconstruction for the number of time steps, ti, where i is from 1 to N.

In another embodiment of the present invention, the image processing method is used to assess quantitative myocardial blood flow and myocardial flow reserve, wherein the input signal enters a multi-layer perceptron (MLP), an artificial neural network and/or convolutional neural network (CNN) and/or long short term memory (LSTM) network to simultaneously predict uptake rate (K1), washout rate (K2) and total blood volume (TBV).

In another embodiment of the present invention, the image processing method is used to assess quantitative myocardial blood flow and/or myocardial flow reserve, wherein the imaging agent or radionuclide is administered by an automated generation and infusion system and/or intravenous administration of radiopharmaceuticals produced by fission, neutron activation, cyclotron, generator and/or combinations thereof.

In another embodiment of the present invention, an image processing method to assess quantitative myocardial blood flow and myocardial flow reserve, comprises the steps of:
a. a step of pre-processing of images comprising:
   (i) reconstructing dynamic cine 3D tomographic myocardial perfusion imaging (MPI) data,
   (ii) isolating value at voxel (i, j, k) for each time point t; where i is from 1 to N,
   (iii) optionally, denoising to improve the quality of image,
   (iv) extracting blood input function from a region of interest (ROI) of the left ventricle blood cavity or other arterial blood region of interest (ROI),
   (v) estimating the distribution volume (DV), given by the ratio of uptake and washout rates (K1/K2) to stabilize and improve estimation of K1, K2 and total blood volume (TBV) and subsequent myocardial blood flow measures, and
   (vi) data normalization by dividing by the maximum of the blood input function,
b. a step of applying the time series at voxel (i, j, k) and blood input function to artificial intelligence network simultaneously to predict uptake K1, K2 and TBV,
c. a step of post-processing of K1, K2 and TBV parametric maps comprising:
   (i) partial volume correction,
   (ii) extraction fraction to estimate myocardial blood flow (MBF) at rest and stress; and
d. a step of post-processing of rest and stress myocardial blood flow to estimate myocardial flow reserve (MFR) and/or coronary flow reserve (CFR);
   wherein the artificial neural networks are selected from the group consisting of, multi-layer perceptron (MLP), artificial neural network (ANN), convolutional neural network (CNN), recurrent neural network (RNN), long short-term memory recurrent neural network (LSTM-RNN), gated recurrent unit (GRU) network, generative adversarial networks (GANs), deep machine learning and/or combinations thereof.

In another embodiment, the present invention includes an image processing method to estimate the arterial input function, wherein the artificial neural network is trained to directly predict one or several pharmacokinetic parameters (e.g., K1, k2, etc.) from a set of ground truth labels.

In another embodiment of the present invention, an image processing method is used to estimate the arterial input function, wherein the artificial neural network is trained to directly predict one or several pharmacokinetic parameters (e.g., K1, k2, etc.) by incorporating the equations of the appropriate compartmental model in order to minimize the error between the theoretical time activity curve (TAC) from said compartment model to the observed TAC at a given voxel.

In another embodiment of the present invention, an image processing method is used to estimate the arterial input function, wherein the artificial neural network can also predict K1, k2, k3, k4 of the three-compartment model (two-tissue compartment model) or K1, k2, k3, k4, k5, k6 of the four-compartment model (three-tissue compartment model) and K1, k2′, k3′, k4 of the four compartment model where k2′=k2/(1+k5/k6) and k3′=k3/(1+k5/k6), as is typically used in brain compartmental modeling for brain receptor studies. All modifications of the aforementioned models, where at least one of these parameters is fixed to equal 0 and is thus not predicted. All aforementioned parameters with delay (Δt) and dispersion factors. All aforementioned parameters with fractional blood pool terms (TBV) and spillover corrections from other blood pools (for example, the right ventricle VRV). These above-mentioned models can also be used for calculation of other parametric map, where the present method of calculation of arterial input function by using unsupervised learning can be used.

### BRIEF DESCRIPTION OF DRAWINGS

Further features and advantages of the present invention will become apparent from the following detailed description, taken in combination with the appended drawings, in which:

FIG. 1 illustrates the flow diagram, wherein the model takes blood input function, and the time series at voxel for output K1, K2 and TBV (total blood volume).

FIG. 2 illustrates the flow diagram, wherein the model takes blood input function, the time series at voxel, and distribution volume (DV) for output K1 and TBV (total blood volume).

FIG. 3 illustrates the flow diagram for model training.

FIGS. 4-19 illustrates the ground truth vs AI-generated parametric maps.

FIG. 20 illustrates a workflow for training the AI-AIF segmentation network.

FIG. 21 illustrates a workflow to use the trained AI-AIF segmentation network for quality assurance of the algorithm and for generating parametric maps used for image interpretation or outcome prediction.

FIG. 22 illustrates AI-derived ROIs superimposed on a representative patient's stress and rest 82Rb exams wherein the ROI is automatically placed on a region spanning the left and right ventricle.

FIG. 23 illustrates agreement between K1 and TBV parametric maps of four test patients' stress images using reference nonlinear least squares (NLS) curve fitting vs. AI. For each panel: (left) NLS reference map, (middle) AI generated, (right) AI-NLS residuals map, top (K1), bottom (TBV).

FIG. 24 illustrates model validation protocol.

FIG. 25 illustrates visual comparison of a representative subject highlighting improved right ventricular blood pool visualization with BV parametric maps versus conventional perfusion-gated PET images.

FIGS. 26A-H illustrates quantitative comparison of conventional gated MPI and AI-generated FBV parametric imaging for EF, SV, EDV, and ESV, validated against cardiovascular magnetic resonance imaging (CMR) measurements.

FIG. 27 illustrates comparative parametric MBF maps, showing significant improvement in CAD detection for TPD at both the global and regional level in comparison with iMFR.

### DETAILED DESCRIPTION OF THE INVENTION

There is an unmet need in the art to improve radioisotope imaging procedures to estimate and/or predict small regional dysfunction or disorders related to the myocardial blood flow (MBF) and/or myocardial flow reserve (MFR), wherein the image series fit to a one-tissue-compartment model yields voxel-wise parametric maps comparing the projected data onto a two-dimensional (2D) polar map of the organ of interest such as left ventricle (LV) myocardium. The inventors of the present invention surprisingly found an advantage in producing regional flow and reserve values, which may better highlight small regional flow defects and are independent of LV polar-map segmentation. The inventors of the present invention found that by using an alternative 3D parametric imaging method of myocardial perfusion with radioisotopes, one can accurately estimate and/or predict the myocardial blood flow (MBF) and/or myocardial flow reserve (MFR). The present invention can be more readily understood by reading the following detailed description of the invention and included embodiments.

As used herein, the articles “a,” “an,” “the,” and “said” are intended to mean that there are one or more of the elements. The terms used herein “comprising,” “including,” and “having” are intended to be inclusive and mean that there may be additional elements other than the listed elements. Further, any numerical examples in the following discussion are intended to be non-limiting, and thus additional numerical values, ranges, and percentages are within the scope of the disclosed embodiments.

As used herein, the term “imaging” refers to techniques and processes used to create images of various parts of the human body for diagnostic and treatment purposes within digital health. X-ray radiography, Fluoroscopy, Magnetic resonance imaging (MRI), Computed Tomography (CT), Medical Ultrasonography or Ultrasound Endoscopy Elastography, Tactile imaging, Thermography Medical photography, and nuclear medicine functional imaging techniques e.g. Positron Emission Tomography (PET), Dynamic Positron Emission Tomography or Single-photon Emission Computed Tomography (SPECT). Imaging seeks to reveal internal structures of the body, as well as to diagnose and treat disease.

As used herein, the term “Positron Emission Tomography (PET)” refers to a functional imaging technique that uses radioactive substances known as radiotracers or radionuclides to visualize and measure changes in metabolic processes, and in other physiological activities including blood flow, regional chemical composition, and absorption. Different tracers can be used for various imaging purposes, depending on the target process within the body; commonly used radionuclide isotopes for PET imaging include Rb-82 (Rubidium-82), Water O-15 (Oxygen-15), F-18 (Fluorine-18), Ga-68 (Gallium-68), Cu-61 (Copper-61), C-11 (Carbon-11), N-13 (Ammonia-13), Co-55 (Cobalt-55), Zr-89 (Zirconium-89), Cu-62, Cu-64, 1-124, Tc-99m (Technetium), T1-201 (Thallium-201), and FDG (Fluorodeoxyglucose). The preferred radionuclide comprises Rb-82 having a half-life of 75 seconds.

As used herein, the term “SPECT” refers to Single-photon emission computed tomography, which is a nuclear medicine tomographic imaging technique using gamma rays and provides true 3D information. This information is typically presented as cross-sectional slices through the patient but can be freely reformatted or manipulated as required. The technique requires delivery of a gamma-emitting radioisotope (a radionuclide) into the patient, normally through injection into the bloodstream. A marker radioisotope is generally attached to a specific ligand to create a radioligand, whose properties bind it to certain types of tissues. This allows the combination of ligand and radiopharmaceutical to be carried and bound to a region of interest in the body, where the ligand concentration is assessed by a gamma camera. SPECT agents include ^{99m}Tc technetium-99m (^{99m}Tc)-sestamibi, and ^{99m}Tc-tetrofosmin), ¹¹¹In, ⁶⁷Ga, ⁶⁸Ga, ²⁰¹Tl (Thallium-201).

As used herein, the term “diagnosis” refers to a process of identifying a disease, condition, or injury from its signs and symptoms. A health history, physical exam, and tests, such as blood tests, imaging, scanning, and biopsies can be used to help make a diagnosis.

As used herein, the term “assessment” refers to a qualitative and/or quantitative assessment of the blood perfusion in a body part or region of interest (ROI).

As used herein, the term “stress agent” refers to agents used to generate stress in a patient or a subject during imaging procedure. The stress agents according to the present invention are selected from vasodilator agent, for example adenosine, adenosine triphosphate and its mimetic, A2A adenosine receptor agonist, for example regadenoson or adenosine reuptake inhibitor dipyridamole, or other pharmacological agents to increase blood flow to the heart, like catecholamines (for example dobutamine, acetyl-choline, papaverine, ergovine, etc.) or other external stimuli to increase blood flow to the heart such as cold-pressor, mental stress or physical exercise.

As used herein, the term “automated infusion system” or “radionuclide generation” and/or “infusion system” or “Rb-82 elution system” refers to a system for generation and/or infusion of a radionuclide or radiotracer and administration into a subject. The automated infusion system comprises radioisotope generator, dose calibrator, computer, controller, display device, activity detector, cabinet, cart, waste bottle, sensors, shielding assembly, alarms or alerts mechanism, tubing, source vial, diluent or eluant, valves. The automated infusion system can be communicatively or electronically coupled to imaging system.

As used herein, the term “dose” refers to the dose of radionuclide required to perform imaging in a subject. The dose of a radionuclide to be administered into the subject ranges from 0.01 MBq to 10,000 MBq.

As used herein, the term “coronary artery disease” or “cardiovascular disease” refers to a disease of major blood vessels. Cholesterol-containing deposits (plaques) in coronary arteries and inflammation are causes of coronary artery disease. The coronary arteries supply blood, oxygen and nutrients to the heart. A buildup of plaque can narrow these arteries, decreasing blood flow to the heart. Eventually, the reduced blood flow may cause chest pain (angina), shortness of breath, or other coronary artery disease signs and symptoms. Significant blockage of the arteries can cause a heart attack. It can be diagnosed by imaging of the myocardium and/or myocardial blood flow (MBF) under rest or pharmacologic stress conditions to evaluate regional myocardial perfusion.

As used herein, the term “myocardial blood flow (MBF)” can be defined as the volume of blood transiting through tissue at a certain rate. MFR constitutes the ratio of MBF during maximal coronary vasodilatation to resting MBF and is therefore impacted by both rest and stress flow. MFR represents the relative reserve of the coronary circulation.

As used herein, the term “pharmacokinetic model” refers to a hypothesis using mathematical terms to describe quantitative relationships and is efficient in describing the time course of the drug throughout the body and is helpful in computing and calculating desired pharmacokinetic parameters, which are needed for achieving the overall objective of drug therapy. The process and kinetics involved in drug distribution and disposition are complex, and drug events often happen simultaneously. The process is governed by a variety of factors that must be properly defined and quantified for designing optimum drug therapy regimens through pharmacokinetic models. The pharmacokinetic model herein of the present invention is used to estimate the pharmacokinetic parameters such as K1, k2, TBV and the like. The term refers herein to the present invention, wherein the pharmacokinetic model can be selected from the group consisting of heart, brain, kidneys, lower extremities and/or combinations thereof.

As used herein, the term “radionuclide” or “radioisotope” refers to an unstable form of a chemical element that releases radiation as it breaks down and becomes more stable. Radionuclides can occur in nature or can be generated in a laboratory. In medicine, they are used in imaging tests and/or in treatment.

As used herein, the term “Sr/Rb elution system” or “⁸²Sr/⁸²Rb elution system” refers to infusion systems meant for generating a solution containing Rb-82, measuring the radioactivity in the solution, and infusing the solution into a subject in order to perform various studies on the subject's region of interest.

As used herein, the term “image counts” refers to number of radioisotope disintegrations acquired per unit time by the PET scanner.

As used herein, the term “generator” or “radioisotope generator” refers to a hollow column inside a radio-shielded container. The column is filled with an ion exchange resin and radioisotope loaded onto the resin. Radionuclide generator according to the present invention is selected from ⁹⁹Mo/^{99m}Tc, ⁹⁰Sr/⁹⁰Y, ⁸²Sr/⁸²Rb, ¹⁸⁸W/¹⁸⁸Re, ⁶⁸Ge/⁶⁸Ga, ⁴²Ar/⁴²K, ⁴⁴Ti/⁴⁴Sc, ⁵²Fe/^{52m}Mn, ⁷²Se/⁷²As, ⁸³Rb/^{83m}Kr, ¹⁰³Pd/^{103m}Rh, ¹⁰⁹Cd/^{109m}Ag, ¹¹³Sn/^{113m}In, ¹¹⁸Te/¹¹⁸Sb, ¹³²Te/¹³²I, ¹³⁷Cs/^{137m}Ba, ¹⁴⁰Ba/¹⁴⁰La, ¹³⁴Ce/¹³⁴La, ¹⁴⁴Ce/¹⁴⁴Pr, ¹⁴⁰Nd/¹⁴⁰Pr, ¹⁶⁶Dy/¹⁶⁶Ho, ¹⁶⁷Tm/^{167m}Er, ¹⁷²Hf/¹⁷²Lu, ¹⁷⁸W/¹⁷⁸Ta, ¹⁹¹Os/^{191m}Ir, ¹⁹⁴Os/¹⁹⁴Ir, ²²⁶Ra/²²²Rn and ²²⁵Ac/²¹³Bi, ⁶⁴Zn/⁶¹Cu.

As used herein, the term “eluant” refers to the liquid or the fluid used for selectively leaching out the daughter radioisotopes from the generator column.

As used herein, the term “eluate” refers to the radioactive eluant after acquisition of daughter radioisotope from the generator column.

As used herein, the term “controller” refers to a computer or a part thereof programmed to perform certain calculations, execute instructions, and control various activities of an elution system based on user input or automatically.

As used herein, the term “activity detector” refers to a component that is used to determine the amount of radioactivity present in eluate from a generator, e.g., prior to the administration of the eluate to the patient.

As used herein, the term “Convolutional Neural Network (CNN)” refers to a system that resembles feed forward neural systems. It is a type of artificial neural network used in time series and image and processing that is specifically designed to process pixel data. In deep learning, a convolutional neural network (CNN/ConvNet) is a class of deep neural networks, most commonly applied to analyze time series as well as natural or medical images. The blood input function is extracted from a region of interest (ROI) using manual or automatic procedures. The mean signal within the ROI is extracted at every time point creating a 1D signal blood input function.

As used herein, the term “Multi-layer perceptron (MLP)” refers to the Multilayer Perceptron, and is an example of an artificial neural network that is used extensively for the solution of a number of different problems, including pattern recognition and interpolation.

As used herein, the term “Recurrent neural network (RNN)” refers to a special type of an artificial neural network adapted to work for time series data or data that involves sequences. Ordinary feed forward neural networks are only meant for data points, which are independent of each other. The RNN method can be Long short-term memory (LSTM) or Gated Recurrent Unit (GRU) network. RNNs can work in conjunction with CNNs to form networks, such as the CNN-LSTM.

As used herein, the term “Gated Recurrent Unit (GRU)” refers to a type of Recurrent Neural Network (RNN) and uses less memory. It is a part of a specific model of recurrent neural network that intends to use connections through a sequence of nodes to perform machine-learning tasks associated with memory and clustering. It has a gating mechanism in recurrent neural networks.

As used herein, the term “voxel” refers to a value on a regular grid in three-dimensional space in three-dimensional (3D) computer graphics. Voxel is short for volume pixel, the smallest distinguishable cube-shaped part of a 3D image. Voxelization is the process of adding depth to an image using a set of cross-sectional images known as a volumetric dataset. These cross-sectional images (or slices) are made up of pixels. Pulling pixels and slices together, a three-dimensional (3D) partition of the image space into volume elements (voxels) forming a 3D scalar field.

As used herein, the term “Tissue Response Function (TRF)” refers to a tracer kinetic modelling, which is used to estimate physiological parameters such as myocardial blood flow (MBF) by mapping or transforming the shape of the “arterial input function (AIF)” to the shape of TRF.

As used herein, the term “blood input function” is commonly known as arterial input function (AIF), which is defined as the concentration of the tracer in an artery measured over time by placing a region of interest.

An embodiment of the present invention includes an image processing method to assess quantitative myocardial blood flow and myocardial flow reserve, comprising the steps of:
a. a step of pre-processing of images comprising:
   (i) reconstructing dynamic cine 3D tomographic myocardial perfusion imaging (MPI) data,
   (ii) isolating value at voxel (i, j, k) for each time point t; where i is from 1 to N,
   (iii) optionally, denoising to improve the quality of image,
   (iv) extracting blood input function from a region of interest (ROI) of the left ventricle blood cavity or other arterial blood region of interest (ROI),
   (v) estimating the distribution volume (DV), given by the ratio of uptake and washout rates (K1/K2) to stabilize and improve estimation of K1, K2 and total blood volume (TBV) and subsequent myocardial blood flow measures, and
   (vi) data normalization by dividing by the maximum of the blood input function
b. a step of assessing the individual signals pre-processed in step (a) in order to generate K1 and TBV parametric maps using artificial neural network;
c. a step of post-processing of K1, K2 . . . Kn, X2, R2, distribution volume (DV) and TBV parametric maps, and of rest and stress myocardial blood flow to estimate myocardial flow reserve (MFR) and/or coronary flow reserve (CFR).

In an embodiment of the present invention, the image processing method is used to assess quantitative myocardial blood flow and myocardial flow reserve, wherein the image reconstruction of arrays is a dynamic series comprising the 3D tomographic voxel (i, j, k) from PET reconstruction for the number of time steps, ti, where i is from 1 to N.

In an embodiment of the present invention includes use of the image processing method to assess quantitative myocardial blood flow and myocardial flow reserve, wherein a region of interest (ROI) can be manual and/or automatic procedures.

In an embodiment of the present invention, the image processing method is used to assess quantitative myocardial blood flow and myocardial flow reserve, wherein the data normalization is performed by dividing by the maximum of the blood input function.

In an embodiment of the present invention, the image processing method is used to assess quantitative myocardial blood flow and myocardial flow reserve, wherein the data normalization for blood input function is performed with the value being from 0 to 1.

In an embodiment of the present invention, the image processing method is used to assess quantitative myocardial blood flow and myocardial flow reserve, wherein the input signal enters a multi-layer perceptron and/or an artificial neural network and/or generative adversarial network (GANs) and/or convolutional neural network (CNN) and/or long short term memory (LSTM) network to simultaneously predict uptake rate (K1), washout rate (K2), distributed volume (DV), and total blood volume (TBV).

In another embodiment of the present invention, the image processing method is used to assess quantitative myocardial blood flow and myocardial flow reserve, wherein the images produced include regional flow and reserve values to highlight small regional flow defects.

In an embodiment of the present invention, the image processing method is used to assess quantitative myocardial blood flow and myocardial flow reserve, wherein the artificial neural networks are selected from the group consisting of multi-layer perceptron (MLP), artificial neural network (ANN), convolutional neural network (CNN), recurrent neural network (RNN), long short-term memory recurrent neural network (LSTM-RNN), gated recurrent unit (GRU) network, deep machine learning and/or combinations thereof.

In another embodiment of the present invention, the image processing method is used to assess quantitative myocardial blood flow and myocardial flow reserve, wherein to estimate distribution volume (DV) artificial neural network enter in multiple layers and wherein the multiple layers can be selected from the group consisting of the initial layer of the network, at an intermediate layer, at the penultimate layer, or combinations thereof.

In an embodiment of the present invention, the image processing method assesses quantitative myocardial blood flow and myocardial flow reserve, wherein the model predicts a K2 (washout rate) value.

In an embodiment of the present invention, the image processing method is used to assess quantitative myocardial blood flow and myocardial flow reserve, wherein anisotropic diffusion filtering is with Gaussian filter.

In an embodiment of the present invention, the image processing method is used to assess quantitative myocardial blood flow and myocardial flow reserve, wherein estimating K1, K2 and total blood volume (TBV) is performed on a voxel-wise basis using 1D signal CNN-LSTM to produce more accurate myocardial blood flow (MBF) estimations.

In an embodiment of the present invention, the image processing method is used to assess quantitative myocardial blood flow and myocardial flow reserve, wherein the images are characterized by administering Rb-82, O-15, N-13, F-18, Cu-62, Tc-99m, Tl-201, and/or combinations thereof.

In an embodiment of the present invention, the image processing method is used to assess quantitative myocardial blood flow and myocardial flow reserve, wherein the images are characterized by administering Rb-82 in rest and stress PET perfusion imaging to highlight small regional flow defects.

In an embodiment of the present invention, the image processing method is used to assess quantitative myocardial blood flow and myocardial flow reserve, wherein the imaging agent or radionuclide is administered by automated generation and infusion system and/or intravenous administration of radiopharmaceuticals produced by fission, neutron activation, cyclotron and/or generator.

In an embodiment of the present invention, the image processing method is used to assess quantitative myocardial blood flow and myocardial flow reserve, wherein an automated radioisotope generation and infusion system comprises Rb-82 elution system.

In another embodiment of the present invention, the image processing method is used to assess quantitative myocardial blood flow and myocardial flow reserve, wherein the images obtained fit to one-tissue-compartment model or multi-tissue compartment model by predicting the value of the ratio of myocardial blood flow stress and myocardial blood flow rest to determine myocardial flow reserve and/or coronary flow reserve and wherein performing an assessment of the obtained images to diagnose disease state includes using multi-layer perceptron (MLP), artificial neural network (ANN), convolutional neural network (CNN), recurrent neural network (RNN), long short-term memory recurrent neural network (LSTM-RNN), gated recurrent unit (GRU) network, deep machine learning, deep neural network, artificial neural network and/or combinations thereof.

In an embodiment of the present invention, the image processing method is used to assess quantitative myocardial blood flow and myocardial flow reserve, wherein the imaging comprises positron emission tomography (PET) imaging, dynamic positron emission tomography, single-photon emission computerized tomography (SPECT), magnetic resonance imaging (MRI), computed tomography (CT), and/or combinations thereof.

In an embodiment of the present invention, an image processing method is used to assess quantitative myocardial blood flow and myocardial flow reserve, comprising the steps of:
a. pre-processing of images comprises:
   (i) reconstructing dynamic cine 3D tomographic myocardial perfusion imaging (MPI) data
   (ii) isolating value at voxel (i, j, k) for each time point t; where i is from 1 to N,
   (iii) optionally, denoising to improve the quality of image,
   (iv) extracting blood input function from a region of interest (ROI) of the left ventricle blood cavity or other arterial blood region of interest,
   (v) estimating the distribution volume (DV), given by the ratio of uptake and washout rates (K1/K2) to stabilize and improve estimation of K1, K2 and total blood volume (TBV) and subsequent myocardial blood flow measures, and
   (vi) data normalization by dividing by the maximum of the blood input function;
b. applying the time series at voxel (i, j, k) and blood input function to artificial intelligence network simultaneously to predict uptake K1, K2 and TBV,
c. post-processing of K1, K2 and TBV parametric maps comprises:
   (vii) partial volume correction,
   (viii) extraction fraction to estimate myocardial blood flow (MBF) at rest and stress; and
d. post-processing of rest and stress myocardial blood flow to estimate myocardial flow reserve (MFR) and/or coronary flow reserve (CFR).

In an embodiment of the present invention includes an image processing method to assess quantitative myocardial blood flow and myocardial flow reserve, comprising the steps of:
e. pre-processing of images comprises:
   (ix) reconstructing dynamic cine 3D tomographic myocardial perfusion imaging (MPI) data,
   (x) isolating value at voxel (i, j, k) for each time point t; where i is from 1 to N,
   (xi) optionally, denoising to improve the quality of image,
   (xii) extracting blood input function from a region of interest (ROI) of the left ventricle blood and other arterial blood region of interest,
   (xiii) estimating the distribution volume (DV), given by the ratio of uptake and washout rates (K1/K2) to stabilize and improve estimation of K1, K2 and total blood volume (TBV) and subsequent myocardial blood flow measures, and
   (xiv) data normalization by dividing by the maximum of the blood input function;
f. applying the time series at voxel (i,joke) and blood input function to artificial intelligence network simultaneously to predict uptake K1, K2 and TBV,
g. post-processing of K1, K2 and TBV parametric maps comprises:
   (xv) partial volume correction,
   (xvi) extraction fraction to estimate myocardial blood flow (MBF) at rest and stress; and
h. post-processing of rest and stress myocardial blood flow to estimate myocardial flow reserve (MFR) and/or coronary flow reserve (CFR);
   wherein the artificial neural networks are selected from the group consisting of, multi-layer perceptron (MLP), artificial neural network (ANN), convolutional neural network (CNN), recurrent neural network (RNN), long short-term memory recurrent neural network (LSTM-RNN), gated recurrent unit (GRU) network, Generative adversarial networks (GANs), deep machine learning and/or combinations thereof.

Another embodiment of the present invention includes an image processing method to assess quantitative myocardial blood flow and myocardial flow reserve, comprising the steps of:
a. pre-processing of images comprises:
   (i) reconstructing dynamic cine 3D tomographic myocardial perfusion imaging (MPI) data,
   (ii) isolating value at voxel (i, j, k) for each time point t; where i is from 1 to N,
   (iii) optionally, denoising to improve the quality of image,
   (iv) extracting blood input function from a region of interest (ROI) of the left ventricle blood cavity or other arterial blood region of interest (ROI),
   (v) estimating the distribution volume (DV), given by the ratio of uptake and washout rates (K1/K2) to stabilize and improve estimation of K1, K2 and total blood volume (TBV) and subsequent myocardial blood flow measures, and
   (vi) data normalization by dividing by the maximum of the blood input function;
b. applying the time series at voxel (i, j, k) and blood input function to artificial intelligence network simultaneously to predict uptake K1 and TBV, wherein the average R2 values are in between 0.9 to 1:
c. post-processing of K1 and TBV parametric maps comprises:
   (vii) partial volume correction,
   (viii) extraction fraction to estimate myocardial blood flow (MBF) at rest and stress; and
d. post-processing of rest and stress myocardial blood flow to estimate myocardial flow reserve (MFR) map and/or coronary flow reserve (CFR) map;
   wherein the artificial neural networks are selected from the group consisting of, multi-layer perceptron (MLP), artificial neural network (ANN), convolutional neural network (CNN) and/or 1D convolutional neural network (1D-CNN), recurrent neural network (RNN), long short-term memory recurrent neural network (LSTM-RNN), gated recurrent unit (GRU) network, generative adversarial networks (GANs), deep machine learning and/or combinations thereof.

Another embodiment of the invention includes a method for computing an arterial input function from region of interest, wherein the artificial neural networks construct a Convolutional Long-Short Term Memory (ConvLSTM)-U-Net to take as input reconstructed 4D dynamic PET data and output a 3D probability map for deriving a weighted average AIF by multiplying the probability map to the PET data for each time frame. The proposed AIF along with tissue time activity curves (TACs) of a target organ as disclosed herein the present invention, the LV were used for kinetic modeling of the 1TCM using a second, previously trained AI model. Unlike classical non-linear least squares regression for kinetic modeling, the second AI model can estimate uptake (K1) and fractional blood volume (FBV) upon inference. The AI-derived K1 and FBV values re-entered the 1TCM in order to generate theoretical TACs and were compared to the observed LV TACs to calculate a mean squared error (MSE) loss function. This end-to-end training architecture allowed the MSE error to be back-propagated through the ConvLSTM-U-Net for model updating. In this way, the ConvLSTM-U-Net learns to derive an AIF specific to the TACs of a target organ without any a priori anatomical knowledge or human annotation.

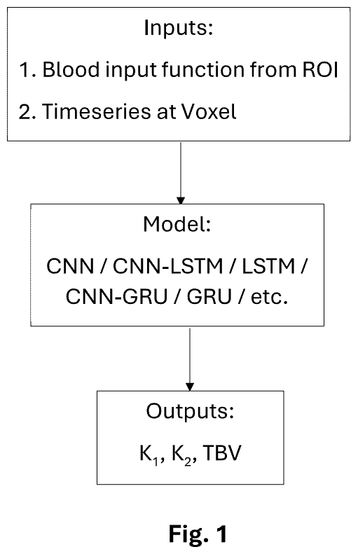

FIG. 1 illustrates the flow diagram, wherein the model takes blood input function, the timeseries at voxel for output K1, K2 and TBV (total blood volume).

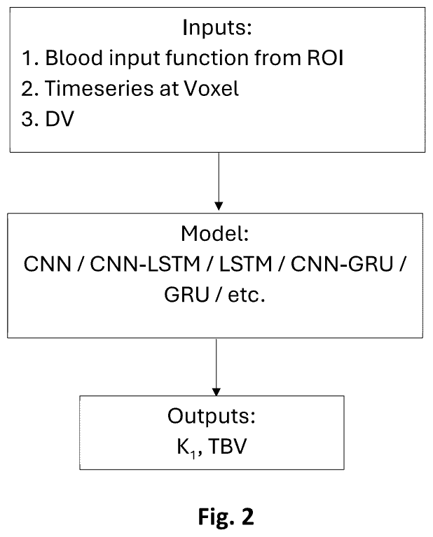

The flow diagram of FIG. 2 illustrates the model that takes blood input function, the timeseries at voxel, distribution volume (DV) for output K1 and TBV (total blood volume).

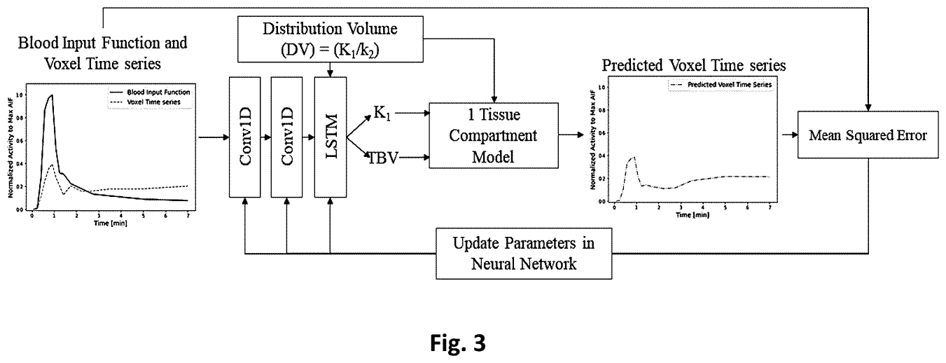

FIG. 3 illustrates the flow diagram for model training, wherein the model takes blood input function, time series at voxel, and distribution volume (DV) for output K1 and TBV (total blood volume). K1, TBV, and DV are inputs to the one tissue compartment model, which yields a predicted voxel time series. The mean squared error between the predicted and input voxel time series is computed and feedback to the model to update model parameters.

Another embodiment of the invention includes, the invention includes a method for computing an arterial input function from region of interest, wherein a neural network model comprising a Convolutional Neural Network (CNN) and Long Short-Term Memory (LSTM) architecture, referred to as a ConvLSTM network. The ConvLSTM model is trained to receive the normalized AIFs and TACs as input and to output voxel-wise kinetic parameters including the uptake rate (K₁), tissue blood volume (TBV), and distribution volume (DV), where DV is defined as the ratio K₁/k₂.

The predicted kinetic parameters are used in a one-tissue compartment model (1TCM) to generate theoretical TACs. The model is trained by minimizing the mean squared error (MSE) between the predicted and observed TACs, thereby optimizing the accuracy of the parameter estimation through backpropagation.

$${C}_{m} ( t ) = TBV \cdot {C}_{a} ( t ) + RC \cdot {K}_{1} {e}^{{-}^{{K}_{1} / {DV}^{\cdot t}}} ⊗ {C}_{a} ( t )$$

wherein, RC is (1-TBV) for LV polar and background TACs; and RC=1 for LV and RV blood pool TACs.

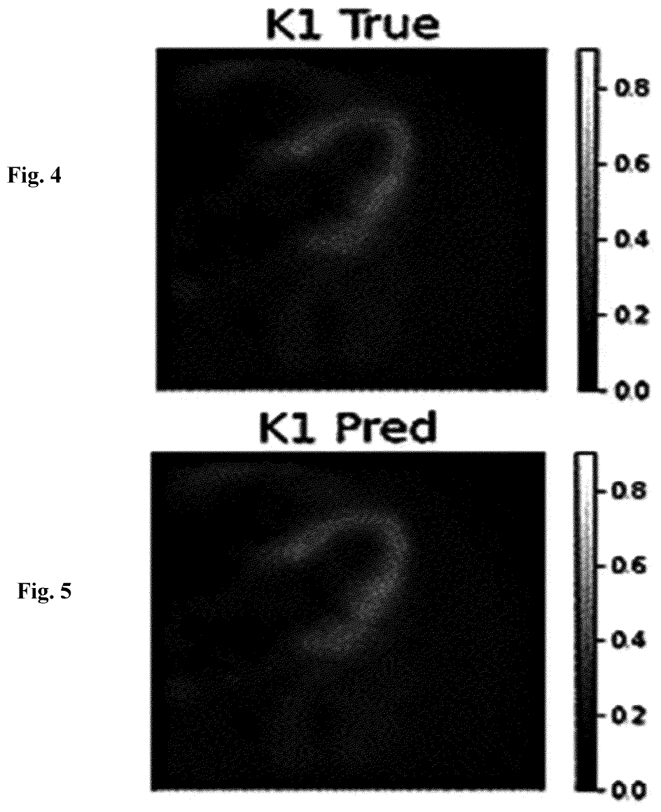

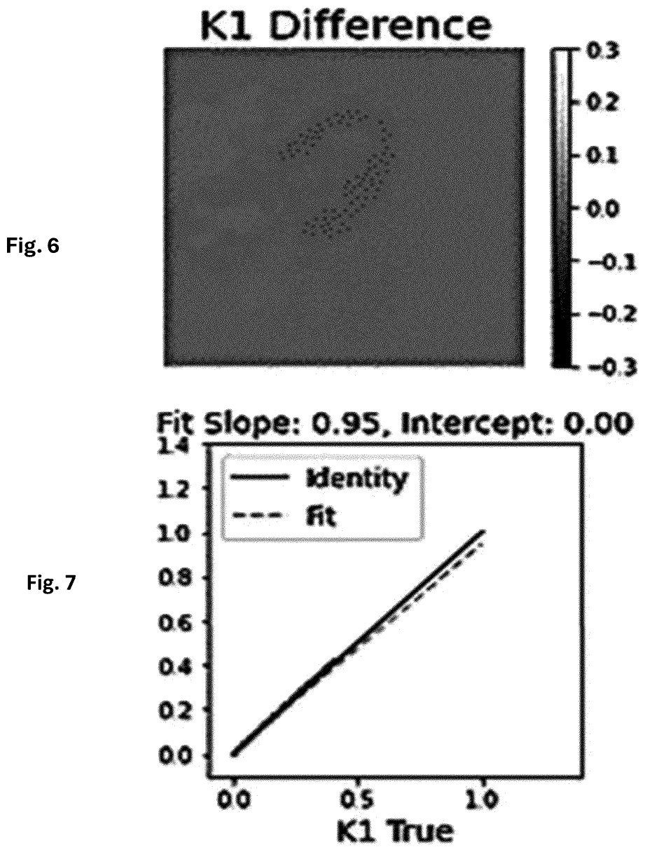

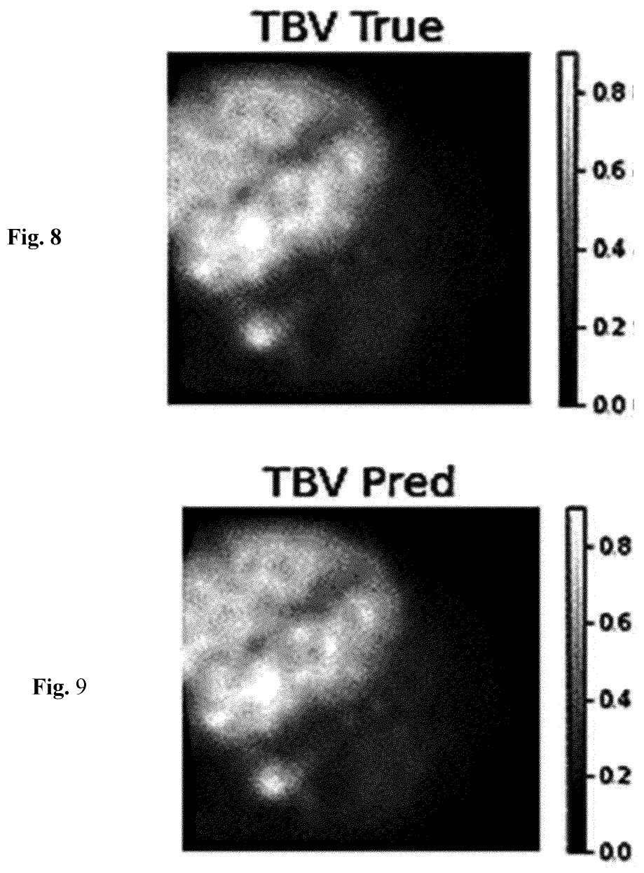

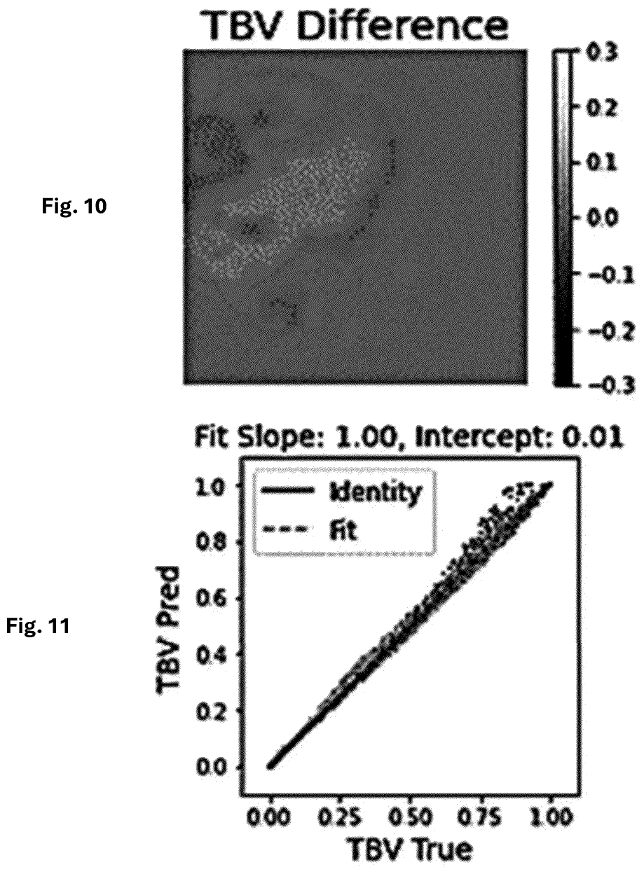

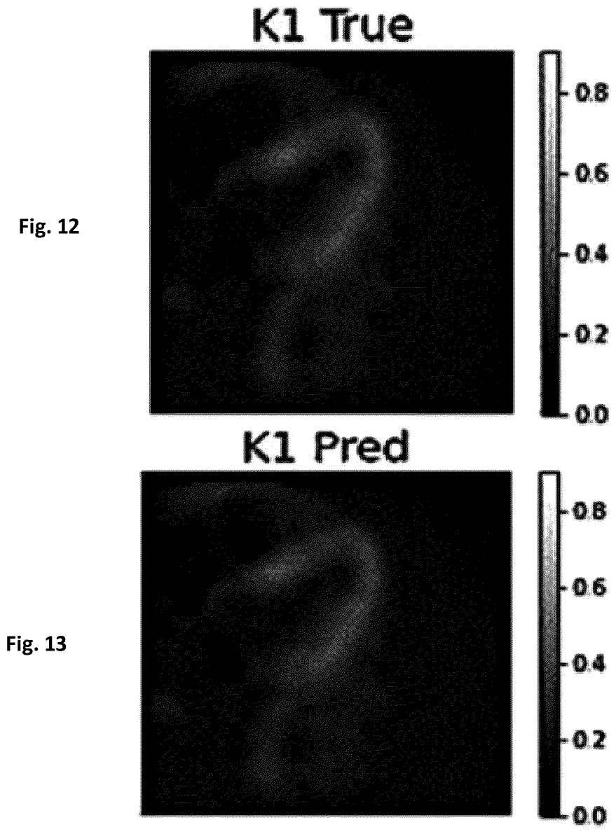

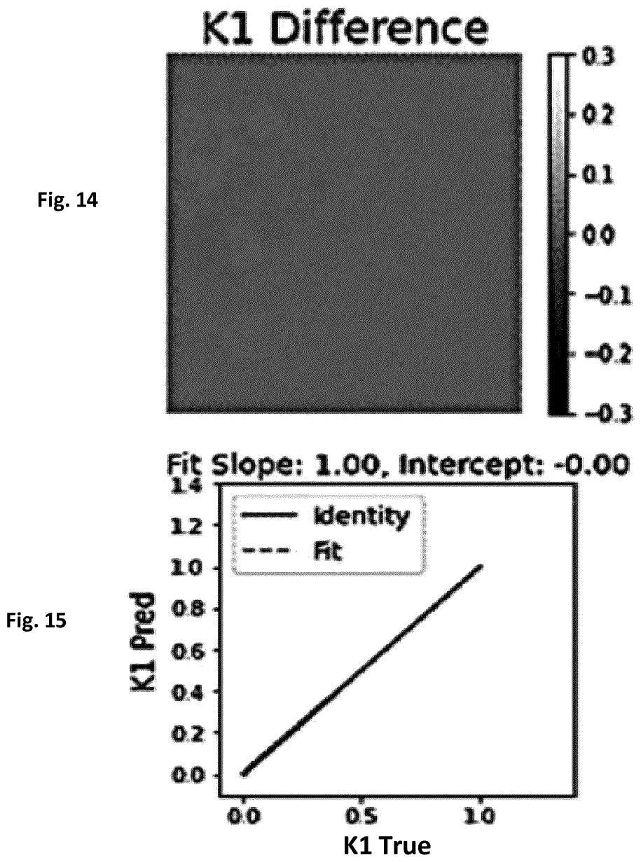

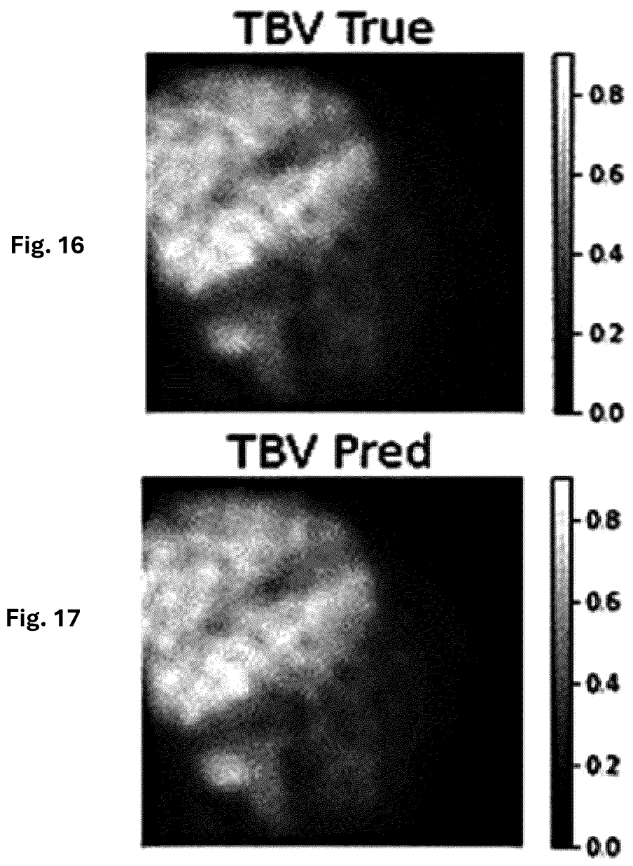

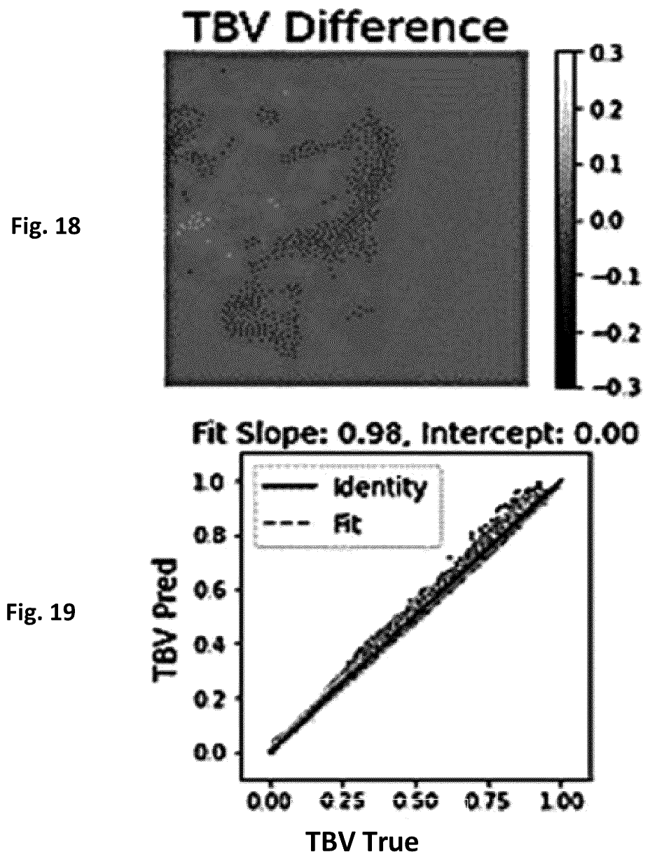

FIGS. 4-19 represent the ground truth vs AI-generated parametric maps. FIGS. 4-11 report the results for a patient's 82Rb Stress scan. Ground Truth K1 parametric maps from non-linear least squares method is depicted as K1 True (shown in FIG. 4), the predicted K1 map from the AI model is depicted as K1 Predicted (K1 15 pred.) (shown in FIG. 5), the difference between K1 True and K1 Pred. is depicted as K1 difference (K1 diff.) (shown in FIG. 6), and a correlation between the whole image K1 True vs K1 Pred. images (shown in FIG. 7). In FIG. 7 the solid line is the identity line and the dotted line is the fit of the correlation. FIGS. 12-19 report the results for a patient's 82Rb rest scan. Ground Truth K1 parametric maps from non-linear 20 least squares method (K1 True) (shown in FIG. 12), the predicted K1 map from the AI model (K1 Pred.) (shown in FIG. 13), the difference between K1 True and K1 Pred. (K1 difference) (shown in FIG. 14), and a correlation between the whole image K1 True vs K1 Pred. images (shown in FIG. 15). In FIG. 15 the solid line is the identity line and the dotted line is the fit of the correlation.

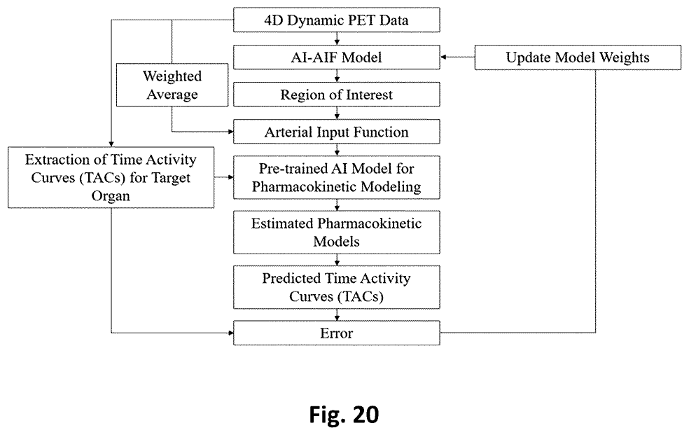

FIG. 20 represents a workflow for training the AI-AIF segmentation network in unsupervised learning way. Obtaining a plurality of dynamic image data sets comprising volumetric image data from region of interest over multiple scanning intervals; utilizing an artificial neural network to segment the plurality of dynamic image data sets displaying one or more arterial input function(s) (AIF) in the region(s) of interest; automatically estimating, using the artificial intelligence, an arterial input function based on a plurality of dynamic image data sets combined with one or more time activity curves (TAC) in the region(s) of interest in target organ(s); computing a pre-trained predictive pharmacokinetic AI model arterial input function using time activity curve input associated with region(s) of interest of target organ(s) and the estimated pharmacokinetic model is used to generate the predicted time activity curves by using the pre-trained pharmacokinetic model.

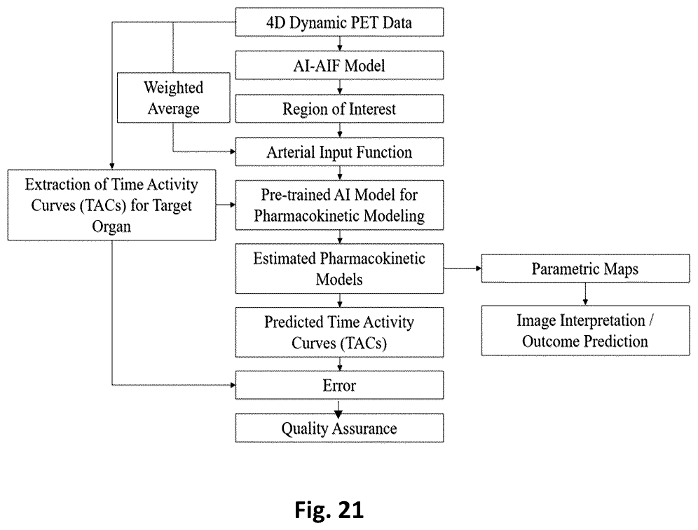

FIG. 21 represents a workflow to use the trained AI-AIF segmentation network for quality assurance of the algorithm and for generating parametric maps used for image interpretation or outcome prediction.

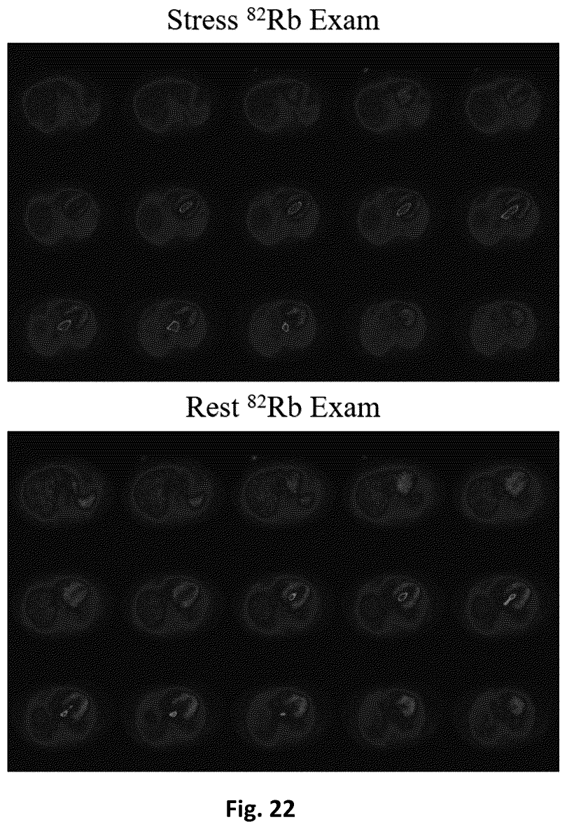

FIG. 22 represents AI-derived ROIs superimposed on a representative patient's stress and rest 82Rb exams wherein the ROI is automatically placed on a region spanning the left and right ventricle.

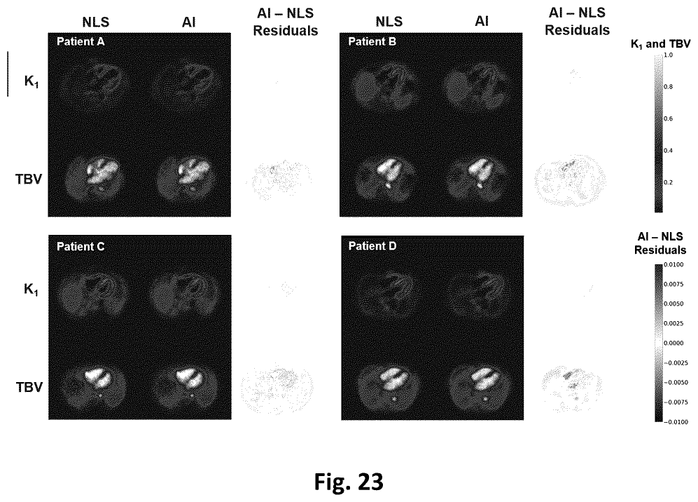

FIG. 23 represents agreement between K1 and TBV parametric maps of four test patients' stress images using reference nonlinear least squares (NLS) curve fitting vs. AI. For each panel: (left) NLS reference map, (middle) AI generated, (right) AI-NLS residuals map, top (K1), bottom (TBV).

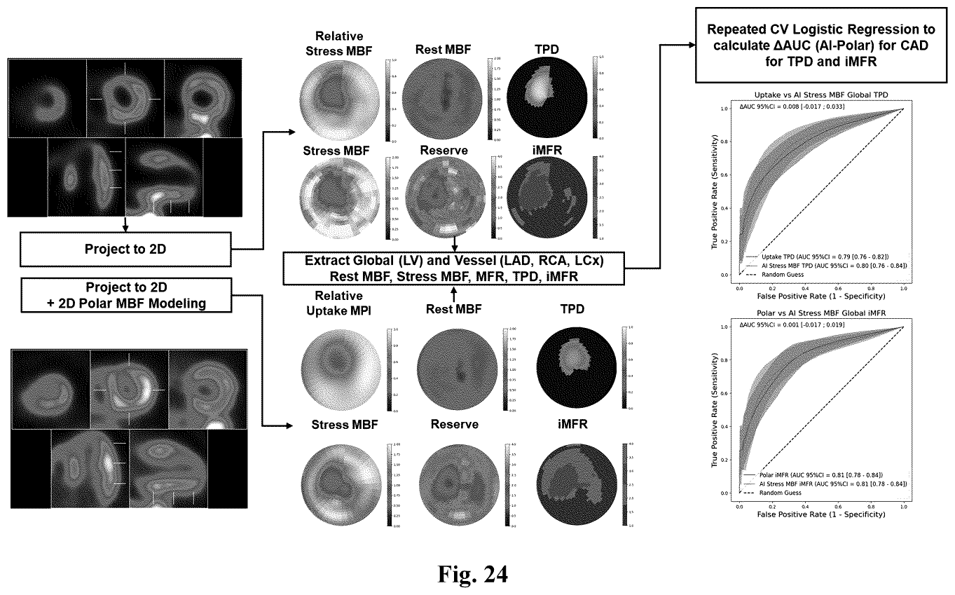

FIG. 24 represents AI model evaluation paradigm. AI-generated MBF maps are projected to polar map space to generate MBF-derived total perfusion deficit (TPD) and focally impaired myocardium extent (iMFR), analogous to conventional polar processing with relative uptake and myocardial flow reserve (MFR). Biomarkers from both methods entered a logistic regression analysis where the area under the curves (AUC) and 95% confidence intervals (CI) were estimated.

An embodiment of the present invention includes use of the image processing method to assess quantitative myocardial blood flow and myocardial flow reserve, wherein consistent image quality is observed in the dose range of Rb-82 is about 1 MBq to about 10,000 MBq.

In another embodiment, the present disclosure provides an artificial intelligence (AI)-based method for generating gated fractional blood volume (BV) parametric maps from gated dynamic ⁸²Rb PET reconstructions. These maps are processed analogously to gated blood pool Single Photon Emission Computed Tomography (SPECT) acquisitions to estimate biventricular parameters including end-diastolic volume (EDV), end-systolic volume (ESV), stroke volume (SV), and ejection fraction (EF).

In an embodiment of the present invention, the image processing method is used to assess quantitative myocardial blood flow and myocardial flow reserve, wherein the imaging comprises X-ray radiography, Fluoroscopy, Magnetic resonance imaging (MRI), Computed Tomography (CT), Medical Ultrasonography or Ultrasound Endoscopy Elastography, Tactile imaging, Thermography Medical photography, and nuclear medicine functional imaging techniques e.g. positron emission tomography (PET), dynamic positron emission tomography, single-photon emission computed tomography (SPECT) imaging and/or combinations thereof.

In an embodiment of the present invention includes the image processing method to assess quantitative myocardial blood flow and myocardial flow reserve, wherein the dose of the imaging agent to be administered is calculated by automated generation and infusion system.

In another embodiment of the present invention, the image processing method is used to assess quantitative myocardial blood flow and myocardial flow reserve, wherein the input signal is enters multi-layer perceptron (MLP), artificial neural network (ANN), convolutional neural network and/or Long Short Term Memory (LSTM) network to simultaneously predict uptake rate (K1), washout rate (K2) and total blood volume (TBV), and wherein performing an assessment of the obtained images to diagnose disease state using deep neural network, artificial neural network, deep machine learning or combinations thereof.

In an embodiment, the trained AI model is applied to new patient data to generate three-dimensional (3D) parametric maps of MBF. These maps are computed globally for the entire left ventricle and regionally for the three major coronary artery territories: the left anterior descending (LAD), left circumflex (LCx), and right coronary artery (RCA).

In one embodiment, the system further derives diagnostic biomarkers from the MBF maps, including myocardial flow reserve (MFR), total perfusion deficit (TPD), and impaired myocardial flow reserve (iMFR). MFR is calculated as the ratio of stress MBF to rest MBF, TPD quantifies the overall reduction in perfusion, and iMFR identifies regions with significantly reduced flow reserve.

In an embodiment, the invention includes a diagnostic evaluation module that applies logistic regression with repeated cross-validation to assess the predictive performance of the derived biomarkers in diagnosing coronary artery disease (CAD), defined as ≥70% stenosis is confirmed from each biomarker used to estimate A AUC (AI-polar) and 95% confidence interval (CI)

In an embodiment, the AI-generated MBF maps are projected into polar map space to facilitate clinical interpretation and compatibility with existing diagnostic workflows. This projection enables direct comparison with conventional 2D polar maps and supports the integration of AI-derived results into standard clinical practice.

In an embodiment, the system demonstrates superior diagnostic performance compared to traditional polar map processing, as validated across multiple clinical centers and independent test datasets. The AI-based method exhibits statistically significant improvements in sensitivity and specificity for detecting CAD, thereby supporting its clinical adoption.

In an embodiment, the system receives dynamic PET imaging data acquired during rest and stress phases of myocardial perfusion using ⁸²Rb as the radiotracer. The imaging data is collected from a plurality of clinical sites and includes a large training dataset. The system extracts arterial input functions (AIFs) and voxel-wise time activity curves (TACs) from the anatomical regions: Left ventricle (LV) cavity, Right ventricle (RV) cavity, Background voxels with low standard deviation, Polar map regions. The extracted AIFs and TACs are interpolated to uniform time intervals and normalized by the maximum value of the AIF to standardize the input data across patients and imaging protocols.

The present invention relates to a deep learning model comprising a hybrid Convolutional Neural Network (CNN) and Long Short-Term Memory (LSTM) architecture, referred to as a ConvLSTM network. This model is configured to receive the pre-processed AIFs and TACs as input and to output voxel-wise kinetic parameters, including: Rate of tracer uptake (K₁), Tissue blood volume (TBV), Distribution volume (DV=K₁/k₂).

These parameters are used in conjunction with a one-tissue compartment model (1TCM) to generate theoretical TACs for each voxel. The model is trained to minimize the mean squared error (MSE) between the predicted and observed TACs, thereby optimizing the accuracy of the kinetic parameter estimation.

The diagnostic accuracy is quantified using the area under the receiver operating characteristic curve (AUC), and the improvement over conventional polar map processing is measured as ΔAUC. The AI-based system demonstrates statistically significant improvements in AUC for TPD and iMFR across both internal and external test datasets.

The invention is designed for seamless integration into clinical workflows. The AI-generated MBF maps are projected into polar map space to facilitate comparison with conventional 2D polar maps. This compatibility ensures that clinicians can interpret AI-derived results using familiar visualization formats while benefiting from enhanced diagnostic sensitivity and specificity.

In an embodiment of the present invention relates to a method and system for non-invasive estimation of biventricular cardiac function using gated dynamic 82Rb PET imaging enhanced by artificial intelligence.

Patients undergo standard clinical ⁸²Rb PET/CT imaging. List-mode PET data are acquired and reconstructed into gated dynamic images using ordered subset expectation maximization (OSEM). The reconstruction includes multiple timeframes (e.g., 9×10 seconds, 3×30 seconds, 1×60 seconds, and 1×120 seconds) and 8 ECG-gated bins to capture cardiac motion.

Dynamic PET images are processed using Jubilant FlowQuant™ software to extract arterial input functions (AIFs), which are essential for downstream parametric mapping. The gated time-activity curves and AIFs are input into a pre-trained convolutional long short-term memory (ConvLSTM) neural network. This network generates gated fractional blood volume (BV) parametric maps that simulate gated blood pool SPECT acquisitions. The BV parametric maps are analyzed using software to estimate biventricular EDV, ESV, SV, and EF. Using CMR as reference, we ran correlation as well as Bland-Altman analyses on percent difference (% DIFF) and compared the mean bias and variance between FBV and MPI using permutation tests.

In a clinical study involving 40 patients who underwent both PET and CMR imaging within a 30-day interval, the AI-generated BV maps demonstrated clear visualization of both left and right ventricular blood pools. Compared to conventional gated MPI, the BV method showed: Reduced variance in right ventricular metrics (>31% improvement), More accurate estimation of left ventricular volumes and EF, Overestimation of LV EF and EDV within acceptable clinical margins, Superior performance in RV assessment, where MPI significantly underestimated ESV and EDV.

The disclosed method demonstrates that AI-generated gated fractional blood volume (BV) parametric imaging using ⁸²Rb PET reliably estimates volumetric and functional parameters including end-diastolic volume (EDV), end-systolic volume (ESV), stroke volume (SV), and ejection fraction (EF) for both the left and right ventricles.

By integrating AI-driven parametric imaging into standard myocardial perfusion and functional assessments within a single imaging modality, ⁸²Rb PET offers a comprehensive and efficient approach to cardiac function evaluation. This advancement extends the diagnostic capabilities of PET imaging to include reliable right ventricular assessment, which has traditionally been limited in conventional PET MPI workflows.

In an embodiment of the present invention the image processing method is used to assess quantitative myocardial blood flow and myocardial flow reserve, wherein administering and performing the test, the steps comprise:
(a) generating a sufficient amount of Rb-82 by automated elution system of Sr-82/Rb-82 radionuclide generator;
(b) administering the generated dose of Rb-82 to the patient;
(c) performing a suitable imaging procedure to obtain better quality images of small regions; and
(d) performing an assessment of the obtained images to diagnose disease state using deep neural network, artificial neural network, deep machine learning, convolutional neural network, recurrent neural network, long short-term memory recurrent neural network (LSTM-RNN), generative adversarial networks (GANs), gated recurrent unit (GRU) network and/or combinations thereof.

An embodiment of the present invention includes using the image processing method to assess quantitative myocardial blood flow and myocardial flow reserve, wherein the method may further comprise administering a stress agent to the subject.

Another embodiment of the present invention includes the image processing method used to assess quantitative myocardial blood flow and myocardial flow reserve, wherein the stress can be induced by administering a stress agent selected from adenosine, adenosine triphosphate, regadenoson, dobutamine, dipyridamole or exercise.

An embodiment of the present invention includes the image processing method used to assess quantitative myocardial blood flow and myocardial flow reserve, wherein the subject weight ranges from 1 kg to 300 kg, preferably in the range of 20 kg to 200 kg.

In an embodiment of the present invention, the image processing method is used to assess quantitative myocardial blood flow and myocardial flow reserve, wherein the automatic dose calculation further comprises other parameters selected from, type of radioisotope, radioisotope half-life, generator life (activity remaining in the radioisotope generator), generator yield, infusion time, flow rate, time lapse from generation to infusion of radioisotope, scanning instrument detector sensitivity, scanner resolution, type of camera or scanner, acquisition time, camera sensitivity, type of disease to be diagnosed, subject conditions like known allergies, heart function, liver function or kidney function or any other special need, subject's supplementary diseases, medications, type of imaging technique to be utilized like PET, SPECT, CT, MRI, and/or combinations thereof.

Another embodiment of the present invention includes the image processing method used to assess quantitative myocardial blood flow and myocardial flow reserve, wherein the automated generation and infusion system comprises a cabinet, radioisotope generator, dose calibrator, computer, controller, display device, activity detector, cabinet, cart, waste bottle, sensors, shielding assembly, alarms or alerts mechanism, tubing, source vial, diluent or eluant, valves or combinations thereof. The automated generation and infusion system generates a radionuclide from a generator/column placed inside the system. A radionuclide eluate is generated from the generator by eluting the generator with suitable eluant like saline, which is then administered by the system automatically after activity measurements. The dose is calculated automatically by the system based on the entered subject parameters. The system is equipped to calculate the flow rate and infusion time depending on the dose to be administered. The automated generation and infusion system can comprise any radionuclide generator, which is suitable for administration to a subject like ⁸²Sr/⁸²Rb generator.

An embodiment of the present invention includes using the image processing method to assess quantitative myocardial blood flow and myocardial flow reserve, wherein the automated generation and infusion system is coupled to the imaging system electronically or communicatively. The coupled imaging system can provide alerts in case image quality is not up to the mark and require repeated administration or scanning.

Another embodiment of the present invention includes using the image processing method to assess quantitative myocardial blood flow and myocardial flow reserve, wherein the automated generation and infusion system is embodied in a portable (or mobile) cart that houses some or all of the generator, the processor, the pump, the memory, the patient line, the bypass line, the positron detector, and/or the calibrator, sensors, dose calibrator, activity detector, waste bottle, controller, display, computer. The cart carrying the components for radioisotope generation and infusion is mobile and can be transferred from one place to another to the patient location or centers, hospitals as required.

An embodiment of the present invention includes using the image processing method to assess quantitative myocardial blood flow and myocardial flow reserve, wherein the method of diagnosing/imaging blood perfusion or flow in the region of interest comprising: input subject parameters into the radioisotope generation and infusion system; automatically calculating the appropriate dose; generating a radionuclide from automated generation or infusion system based on required dose to be administered; administering the radionuclide to the subject in need thereof; performing PET or SPECT scanning of the region of interest; automated analysis of the images by computerized software; quantitative assessment of the blood flow in the region of interest; generating automated report of the assessment; providing appropriate therapy options for the subject.

Another embodiment of the present invention includes using the image processing method to assess quantitative myocardial blood flow and myocardial flow reserve, wherein the subject is a human subject. The human subject is a male or female subject. The age of the subject may vary from 1 month to 120 years. The human subject includes neonates, pediatric, adults and/or geriatric population.

Another embodiment of the present invention includes using the image processing method to assess quantitative myocardial blood flow and myocardial flow reserve, wherein all numbers disclosed herein can vary by 1%, 2%, 5%, 10%, or up to 20% if the word “about” is used in connection therewith. This variation may be applied to all numbers disclosed herein.

Another embodiment of the present invention includes using the image processing method to assess quantitative myocardial blood flow and myocardial flow reserve, wherein 3D parametric images of MBF generated by present invention has better image quality and pixel. This invention also provides a recommendation alert to the medical staff regarding the detection of the coronary diseases by analyzing the generated 3D parametric images of MBF and/or MFR.

Another embodiment of the present invention includes using the image processing method to assess quantitative myocardial blood flow and/or myocardial flow reserve, wherein 3D parametric images of MBF generated by present invention also recommend the calcium scoring. The coronary artery calcium (CAC) score reflects the total area of calcium deposits and the density of the calcium. A score of zero means no calcium is seen in the heart. It suggests a low chance of developing a heart attack in the future. When calcium is present, the higher the score, the higher the risk of heart disease. To evaluate the accuracy and reproducibility of visual estimation of coronary artery calcium (CAC) positron emission tomography (PET), dynamic positron emission tomography (PET), hybrid positron emission tomography (PET), computed tomography (CT) and single-photon emission computed tomography (SPECT)/CT myocardial perfusion imaging (MPI) scans are performed.

Another embodiment of the present invention includes using the image processing method to assess quantitative myocardial blood flow and/or myocardial flow reserve, wherein the image reconstruction algorithms have been developed to improve the quality of images and using the AI algorithm to enhance the image reconstruction quality, which is intended to do the image processing faster and reduce the doses of nuclear medicine up to 10 times during the myocardial perfusion imaging (MPI).

Another embodiment of the present invention includes using the image processing method for AI models to generate blood flow parametric maps with high accuracy and in a timeframe acceptable for clinical use, which may enable future clinical implementation.

Another embodiment of the present invention includes using the image processing method to automatically compute an arterial input function from region of interest, the method comprising:
a. obtaining a plurality of dynamic image data sets comprising volumetric image data from region of interest over multiple scanning intervals;
b. utilizing an artificial neural network to segment the plurality of dynamic image data sets displaying one or more arterial input function(s) (AIF) in the region(s) of interest;
c. automatically estimating, using artificial intelligence, an arterial input function based on plurality of dynamic image data sets combined with one or more-time activity curves (TAC) in the region(s) of interest in target organ(s); and
d. computing a pre-trained predictive pharmacokinetic AI model arterial input function using time activity curve input associated with region(s) of interest of target organ(s).

Another embodiment of the present invention includes using the image processing method to automatically compute an arterial input function from region of interest, wherein the artificial neural network is a self-trained or un-supervised machine learning model.

Another embodiment of the present invention includes using the image processing method to automatically compute an arterial input function from region of interest, wherein artificial neural networks is selected from the group consisting of multi-layer perceptron (MLP), artificial neural network (ANN), convolutional neural network (CNN) and/or 1D convolutional neural network (1D-CNN), recurrent neural network (RNN), long short-term memory recurrent neural network (LSTM-RNN), gated recurrent unit (GRU) network, generative adversarial networks (GANs), deep machine learning, reinforcement learning algorithm and/or combinations thereof.

Another embodiment of the present invention includes using the image processing method to automatically compute an arterial input function from region of interest, wherein the pre-trained predictive pharmacokinetic AI model is used the estimate the pharmacokinetic parameters.

Another embodiment of the present invention includes using the image processing method to automatically compute an arterial input function from region of interest, wherein the pharmacokinetic modelling can be selected from the group consisting of one, two, three, or four tissue compartment model.

Another embodiment of the present invention includes using the image processing method to automatically compute an arterial input function from region of interest, wherein the pharmacokinetic AI model can be selected from the group consisting of heart, brain, kidneys, lower extremities and/or combinations thereof.

Another embodiment of the present invention includes using the image processing method to automatically compute an arterial input function from region of interest, wherein the one, two, three, or four tissue compartment model estimates the K1, k2, fractional blood volume, total blood volume and/or combinations thereof.

Another embodiment of the present invention includes using the image processing method to automatically compute an arterial input function from region of interest, wherein the image data is characterized by administering Rb-82, O-15, N-13, Cu-62-PTSM, 99m-Tc-Sestamibi, Tl-201, and/or combinations thereof.

Another embodiment of the present invention includes using the image processing method to automatically compute an arterial input function from region of interest wherein the image-data is characterized by administering Rb-82 in rest and stress PET perfusion imaging to highlights small regional flow defects.

Another embodiment of the present invention includes using the image processing method to automatically compute an arterial input function from region of interest, wherein the imaging agent or radionuclide is administered by automated generation and infusion system and/or intravenous administration of radiopharmaceuticals produced by fission, neutron activation, cyclotron and/or generator.

Another embodiment of the present invention includes using the image processing method to automatically compute an arterial input function from region of interest, wherein automated radioisotope generation and infusion system comprises Rb-82 elution system.

Another embodiment of the present invention includes using the image processing method to automatically compute an arterial input function from region of interest, wherein the pre-trained predictive pharmacokinetic AI model is a self-trained or un-supervised machine learning model.

Another embodiment of the present invention includes using the image processing method to automatically compute an arterial input function from region of interest, wherein the method further comprises using the error of the predicted time activity curves from the observed time activity curves in the region of interest (ROI) for quality assurance.

Another embodiment of the present invention includes using the image processing method to automatically compute an arterial input function from region of interest, wherein the error is mean squared error (MSE) with a threshold value and mean squared error (MSE) is used to determine the reliability of the region of interest (ROI) and derived AIF.

Another embodiment of the present invention includes using the image processing method to automatically compute an arterial input function (AIF) from region of interest (ROI), wherein the method further comprises generating a parametric map using a trained AIF-ROI segmentation.

Another embodiment of the present invention includes using the image processing method to automatically compute an arterial input function (AIF) from region of interest (ROI), wherein the method further comprises generating a parametric maps using a trained AIF-ROI segmentation in combination with one or more parametric mapping method.

Another embodiment of the present invention includes using the image processing method to automatically compute an arterial input function (AIF) from region of interest (ROI), wherein the one or more parametric mapping methods can be selected from the group of nonlinear least squares regression, basis function method, AI-based model for pharmacokinetic modelling or combinations thereof.

Another embodiment of the present invention includes using the image processing method to automatically compute an arterial input function (AIF) from region of interest (ROI), the method comprising:
a. obtaining a plurality of dynamic image data sets comprising volumetric image data from region of interest over multiple scanning intervals;
b. utilizing an artificial neural network to segment the plurality of dynamic image data sets displaying one or more arterial input function(s) (AIF) in the region(s) of interest (ROI);
c. automatically estimating, using artificial intelligence, an arterial input function (AIF) based on plurality of dynamic image data sets combined with one or more-time activity curves (TAC) in the region(s) of interest (ROI) in target organ(s); and
d. computing a pre-trained predictive pharmacokinetic AI model arterial input function using time activity curve (TAC) input associated with region(s) of interest (ROI) of target organ(s);
   wherein the pre-trained predictive pharmacokinetic AI model is used for estimating the associated parameter to determine the parametric map.

In an embodiment of the present invention includes using the image processing method to automatically compute an arterial input function (AIF) from region of interest (ROI) method comprising:
a. obtaining a plurality of dynamic image data sets comprising volumetric image data from region of interest (ROI) over multiple scanning intervals;
b. utilizing an artificial neural network to segment the plurality of dynamic image data sets displaying one or more arterial input function(s) (AIF) in the region(s) of interest (ROI);
c. automatically estimating, using the artificial intelligence, an arterial input function (AIF) based on plurality of dynamic image data sets combined with one or more-time activity curves (TAC) in the region(s) of interest (ROI) in target organ(s); and
d. computing a pre-trained predictive pharmacokinetic AI model arterial input function using time activity curve (TAC) input associated with region(s) of interest (ROI) of target organ(s);
   wherein the pre-trained predictive pharmacokinetic AI model is used for estimating the associated parameter like K1, K2 and TBV to estimate myocardial flow reserve (MFR) map and/or coronary flow reserve (CFR) map.

In an embodiment of the present invention includes a system comprises a method for computing myocardial blood flow (MBF) and related biomarkers using artificial intelligence, comprising:
a. receiving arterial input functions (AIFs) and voxel time activity curves (TACs);
b. feeding the AIFs and voxel TACs into a convolutional long short-term memory neural network (ConvLSTM);
c. predicting kinetic parameters including K1, total blood volume (TBV), and distribution volume (DV) using the ConvLSTM;
d. generating theoretical TACs using a one tissue compartment model (1TCM) based on the predicted parameters; and
e. optimizing the ConvLSTM model by minimizing the mean squared error (MSE) between observed and theoretical TACs.

In an embodiment of the present invention includes the system, wherein the distribution volume (DV) is computed as the ratio of K1 to k2.

In an embodiment of the present invention includes a system comprises a method for computing myocardial blood flow (MBF) and related biomarkers using artificial intelligence, comprising:
a. receiving arterial input functions (AIFs) and voxel time activity curves (TACs);
b. feeding the AIFs and voxel TACs into a convolutional long short-term memory neural network (ConvLSTM);
c. predicting kinetic parameters including K1, total blood volume (TBV), and distribution volume (DV) using the ConvLSTM;
d. generating theoretical TACs using a one tissue compartment model (1TCM) equation:

$${C}_{m} ( t ) = TBV \cdot {C}_{a} ( t ) + RC \cdot {K}_{1} {e}^{{-}^{{K}_{1} / {DV}^{\cdot t}}} ⊗ {C}_{a} ( t ) ;$$

wherein, RC is (1-TBV) for LV polar and background TACs; and RC=1 for LV and RV blood pool TACs; and
e. optimizing the ConvLSTM model by minimizing the mean squared error (MSE) between observed and theoretical TACs.

In an embodiment of the present invention includes the system, wherein the ConvLSTM model is trained using repeated cross-validation.

In an embodiment of the present invention includes the system, wherein the theoretical TACs are generated voxel-wise.

In an embodiment of the present invention includes the system, wherein the mean squared error (MSE) back-propagation includes optimization through the ConvLSTM network.

In an embodiment of the present invention includes the system, wherein the method further comprises extracting global (LV) and regional MBF and myocardial flow reserve (MFR) from AI-MBF maps and polar processing.

In an embodiment of the present invention includes the system, wherein global (LV) and regional MBF includes left anterior descending (LAD), left circumflex (LCx), and right coronary artery (RCA) territories, and reverse MFR are extracted for AI-MBF maps and polar processing.

In an embodiment of the present invention includes a system for diagnosing coronary artery disease (CAD) using AI-derived myocardial biomarkers, comprising:
a. a ConvLSTM neural network configured to predict K1, TBV, and DV from AIFs and voxel TACs;
b. a module for generating theoretical TACs using the 1TCM equation:

$${C}_{m} ( t ) = TBV \cdot {C}_{a} ( t ) + RC \cdot {K}_{1} {e}^{{-}^{{K}_{1} / {DV}^{\cdot t}}} ⊗ {C}_{a} ( t ) ;$$

wherein, RC is (1-TBV) for LV polar and background TACs; and RC=1 for LV and RV blood pool TACs;
a. a polar map projection module for converting AI-generated MBF maps into polar space;
b. a biomarker extraction module configured to compute total perfusion deficit (TPD) and integrated myocardial flow reserve (iMFR); and
c. a logistic regression module configured to predict CAD and estimate area under the curve (AUC) and confidence intervals (CI) from the extracted biomarkers.

In an embodiment of the present invention includes the system, wherein the polar map projection module uses AI-MBF maps to compute TPD and iMFR.

In an embodiment of the present invention includes the system, wherein the logistic regression module uses biomarkers from both AI-derived and conventional polar processing methods.

In an embodiment of the present invention includes the system, wherein the logistic regression module estimates AUC and CI for predicting CAD with ≥70% stenosis.

In an embodiment of the present invention includes the system, wherein the ConvLSTM neural network is configured to receive both AIFs and voxel TACs as time-series inputs.

In an embodiment of the present invention includes the system, wherein the biomarker extraction module computes focally impaired myocardial extent from iMFR.

In an embodiment of the present invention includes the system, wherein the polar map projection module performs analogous processing to conventional relative uptake methods.

In an embodiment of the present invention includes a system comprises a method for estimating biventricular cardiac function using 82Rb positron emission tomography (PET), comprising:
a. acquiring gated dynamic PET imaging data of a subject heart;
b. reconstructing the gated dynamic PET data into time-resolved images;
c. extracting arterial input functions (AIFs) from the reconstructed images;
d. inputting the gated time activity curves and AIFs into a pre-trained convolutional long short-term memory (ConvLSTM) neural network;
e. generating gated fractional blood volume (FBV) parametric maps from the neural network; and
f. estimating end-diastolic volume (EDV), end-systolic volume (ESV), stroke volume (SV), and ejection fraction (EF) for both left and right ventricles from the FBV parametric maps.

In an embodiment of the present invention includes the system, wherein the dynamic PET imaging data is acquired using a list-mode protocol and reconstructed using ordered subset expectation maximization (OSEM) with multiple time frames and ECG-gated bins.

In an embodiment of the present invention includes the system, wherein the fractional blood volume (FBV) parametric maps are processed using software to derive biventricular functional parameters.

In an embodiment of the present invention includes the system, wherein the ConvLSTM neural network is trained on gated PET images and corresponding cardiovascular magnetic resonance (CMR) measurements.

In an embodiment of the present invention includes the system, wherein the fractional blood volume (FBV) parametric maps provide enhanced visualization of right ventricular blood pools compared to conventional gated myocardial perfusion imaging (MPI).

In an embodiment of the present invention includes the system, wherein the estimated biventricular parameters are validated against CMR-derived measurements using correlation and Bland-Altman analyses. Each embodiment disclosed herein is contemplated as being applicable to each of the other disclosed embodiments. Thus, all combinations of the various elements described herein are within the scope of the invention.

This invention will be better understood by reference to the experimental data, which follow, but those skilled in the art will readily appreciate that the specific experiments detailed are only illustrative of the invention as described more fully in the claims, which follow thereafter.

### EXPERIMENTAL METHOD

### Example 1

Rb-82 is administered to patients. 20 subjects/patients (N=20) scans are selected with a wide range of uptake defect severities on Rb-82 stress PET perfusion imaging. The input signal is multi-layer perceptron (MLP), artificial neural network (ANN), convolutional neural network (CNN), recurrent neural network (RNN), long short-term memory recurrent neural network (LSTM-RNN), gated recurrent unit (GRU) network, Generative adversarial networks (GANs), deep machine learning and/or combinations thereof network to simultaneously predict uptake rate (K1), K2 and total blood volume (TBV) and the 3D parametric images of K1, K2 and TBV are combined to estimate the MBF and/or MFR.

### Example 2

Rb-82 is administered to 40 patients (N=40) from two scanners (20 from GE Discovery 690, 20 from GE Discovery 600) were identified from Cardiac PET studies from 2019 covering a wide range of defect severities on 82Rb stress PET. Data from the Discovery 690 was split into training/validation/test sets with a 60:20:20 split. All Discovery 600 data constituted a separate hold-out test set. Image-derived arterial blood input functions (AIF) and voxel time series/time activity curves (TACs) in a 196×196×98 mm3 region around the heart were used for this study. Kinetic modeling is performed with one tissue compartment model (1TCM) with the classical nonlinear least squares (NLS) method to produce reference parametric maps. AIFs and voxel TACs were fed to a Convolutional/Long-Short Term Memory Neural Network (CNN-LSTM) to predict K1 and TBV and the associated predicted TACs. The AI model was optimized to minimize the mean squared error between the input and predicted TACs (FIG. 3). The results are depicted below:

### Results

The AI model yielded accurate predictions of K1 and TBV with average R2 values of 0.998 and 0.991 for the Discovery 690, and 0.995 and 0.997 for the Discovery 600 hold-out test sets (FIGS. 4-19). Generating parametric maps on a typical central processing unit (CPU) took an average of 89.1 minutes for the classical NLS method and 7.21 seconds with the AI enabled model as described in Example 2, which is 741 times faster.

These two working examples of the present invention for AI models can generate blood flow parametric maps with high accuracy and in a timeframe acceptable for clinical use and thus may enable future clinical implementation.

### Example 3

### Data Preparation

N=1912 patients from two scanners (918 from a GE Discovery 600, 994 from a GE Discovery 690) were identified from Cardiac PET studies from 2019 at the Ottawa Heart Institute covering a wide range of defect severities on 82Rb stress PET. Data from the Discovery 600 was split into 60:20:20 training: validation: test sets. All Discovery 690 data constituted a separate hold-out test set. Image-derived arterial blood input functions (AIF) and voxel time activity curves (TACs) of the left ventricle (LV) myocardium, cavity, and immediate environment were extracted using the FlowQuant software. AIFs and voxel TACs were normalized by the AIF max.

The inventors of the present invention performed kinetic modeling of the 1TCM with the classical nonlinear least squares (NLS) method to produce reference parametric maps.

### Model Training and Evaluation

AIFs and voxel TACs were fed to a Convolutional/Long-Short Term Memory Neural Network to predict K1 and TBV and the associated theoretical TACS according to the 1TCM. The AI model was optimized to minimize the mean squared error (MSE) between the observed and theoretical TACS (FIG. 1). Finally, the inventors of the present invention computed and compared the average time to compute the parametric maps from a full dynamic 3D acquisition between the AI and classical curve fitting method.

### Results

### Model Training and Performance

The model trained for 40 epochs and achieved the lowest validation MSE loss on the 31st.

Regarding the generation time of parametric maps, AI took an average of 7.21±0.61 seconds vs 89.1±3.2 minutes for classical curve fitting, resulting in an acceleration factor of 741.

The trained model yielded highly accurate predictions of K1 and TBV and showed no signs of overfitting and excellent generalization to unseen patients and data from a different scanner (Table 1, FIG. 23):

TABLE 1
AI model performance on the mean squared error (MSE)
between the observed and theoretical time activity
curves (TACs), the coefficient of determination (R2) for the
reference and predicted TACs, K1, and TBV

| | | | Test | Test |
|---|---|---|---|---|
| Metric | Training | Validation | (Discovery 600) | (Discovery 690) |
| MSE | 0.00165 | 0.00138 | 0.00142 | 0.00160 |
| R²TAC | 0.918 | 0.922 | 0.924 | 0.931 |
| R² K₁ | 0.988 | 0.997 | 0.999 | 0.997 |
| R²TBV | 0.993 | 0.995 | 0.999 | 0.998 |

### Example 4

The dataset comprised of N=2790 patients who underwent rest and stress 82Rb Cardiac PET studies on either a GE Discovery 690 or 600 at the Ottawa Heart Institute for suspicion of coronary artery disease during 2018 and 2019. Data from 2018 constituted the training set (N=3568 scans), whereas the validation (N=958) and test sets (N=1054) were from the Discovery 690 and 600 in 2019, respectively. Constructing a Convolutional Long-Short Term Memory (ConvLSTM)-U-Net to take as input reconstructed 4D dynamic PET data and output a 3D probability map for deriving a weighted average AIF by multiplying the probability map to the PET data for each time frame. The proposed AIF along with tissue time activity curves (TACs) of a target organ—here, the LV—were used for kinetic modeling of the 1TCM using a second, previously trained AI model. Unlike classical non-linear least squares regression for kinetic modeling, the second AI model can estimate uptake (K1) and fractional blood volume (FBV) upon inference. The AI-derived K1 and FBV values re-entered the 1TCM in order to generate theoretical TACs and were compared to the observed LV TACs to calculate a mean squared error (MSE) loss function. This end-to-end training architecture allowed for the MSE error to be back-propagated through the ConvLSTM-U-Net for model updating. In this way, the ConvLSTM-U-Net learns to derive an AIF specific to the TACs of a target organ without any a priori anatomical knowledge or human annotation.

### Results

The ConvLSTM-U-Net model successfully converged after 20 epochs, showing

great generalization to the validation and test sets (MSEtrain=0.00098, MSEval=0.0011, MSEtest=0.00090) and excellent correlation of predicted vs. observed TACs (R2val=0.92, R2val=0.93). The ConvLSTM-U-Net consistently and specifically generated ROIs at the base of the LV and in the LA, concordant with common physiological assumptions of arterial blood inputs.

The self-supervised AI model of the present invention naturally found that the most optimal ROI placement for deriving an AIF for MBF estimate is at the border of the LV and LA. Further, the robust model, trained and validated on a large sample size of the present invention can increase confidence in downstream MBF estimation through interpretable and consistently generated AIFs. Future works will extend this framework for deriving other organ-specific AIFs, such as for the brain, kidneys, etc., and other pharmacokinetic models.

### Example 5

The system processes dynamic PET imaging data acquired during both rest and stress phases of myocardial perfusion using 82Rb as the radiotracer. This data is collected from multiple clinical sites and includes a large training dataset comprising 5,614 patients, along with two independent test datasets of 1,731 and 1,589 patients respectively. From this imaging data, the system extracts arterial input functions (AIFs) and voxel-wise time activity curves (TACs) from specific anatomical regions including the left ventricle (LV) cavity, right ventricle (RV) cavity, background voxels with low standard deviation, and polar map regions. These AIFs and TACs are then interpolated to uniform time intervals and normalized by the maximum value of the AIF to ensure consistency across different patients and imaging protocols. The core of the invention is a deep learning model based on a hybrid architecture combining Convolutional Neural Networks (CNN) and Long Short-Term Memory (LSTM) networks, referred to as a ConvLSTM model. This model takes the pre-processed AIFs and TACs as input and outputs voxel-wise kinetic parameters such as the rate of tracer uptake (K₁), tissue blood volume (TBV), and distribution volume (DV), which is calculated as K₁/k₂. These parameters are used within a one-tissue compartment model (1TCM) to generate theoretical TACs for each voxel, and the model is trained to minimize the mean squared error (MSE) between predicted and observed TACs to enhance the accuracy of kinetic parameter estimation. The results are depicted below:

### Results

The AI model is applied to new PET imaging data to generate three-dimensional parametric maps of myocardial blood flow (MBF). These maps provide both absolute and relative flow values and are computed globally for the entire left ventricle as well as regionally for the three major coronary artery territories: Left Anterior Descending (LAD), Left Circumflex (LCx), and Right Coronary Artery (RCA). From these parametric maps, the system derives clinically significant biomarkers including Myocardial Flow Reserve (MFR), which is the ratio of stress MBF to rest MBF; Total Perfusion Deficit (TPD), a quantitative measure of global perfusion abnormality; and Impaired Myocardial Flow Reserve (iMFR), which indicates the extent of myocardium with reduced flow reserve. A diagnostic module is integrated into the system to evaluate the presence of coronary artery disease (CAD), defined as a stenosis of 70% or more confirmed by invasive coronary angiography (ICA). The system uses logistic regression with repeated cross-validation to assess the predictive performance of each biomarker. Diagnostic accuracy is quantified using the area under the receiver operating characteristic curve (AUC), and improvements over conventional polar map processing are measured as ΔAUC. The AI-based system demonstrates statistically significant improvements in AUC for both TPD and iMFR across internal and external test datasets, highlighting its enhanced diagnostic capabilities.

### Example 6: Validation of AI-Generated Gated Fractional Blood Volume Parametric Maps using ⁸²Rb PET for Estimation of Biventricular Volumes and Function

The method was validated using forty patients who underwent both clinical 82Rb PET/CT and CMR imaging within a 30-day interval at a single center. CMR examinations were conducted using a 3T scanner, with manual delineation of end-diastolic and end-systolic endocardial contours for both ventricles on short-axis cine sequences, following Society for Cardiovascular Magnetic Resonance (SCMR) guidelines.

For PET imaging, list-mode data were acquired and reconstructed into gated dynamic images using Siemens e7 tools. The reconstruction employed ordered subset expectation maximization (OSEM) with timeframes of 9×10 seconds, 3×30 seconds, 1×60 seconds, and 1×120 seconds, and 8 ECG-gated bins. Jubilant FlowQuant™ software was used to extract arterial input functions (AIFs) from the dynamic images.

The gated time-activity curves and AIFs of the present invention were input into a pre-trained convolutional long short-term memory (ConvLSTM) neural network to generate gated BV parametric images. Biventricular EDV, ESV, SV, and EF were then estimated from these maps using software.

### Results

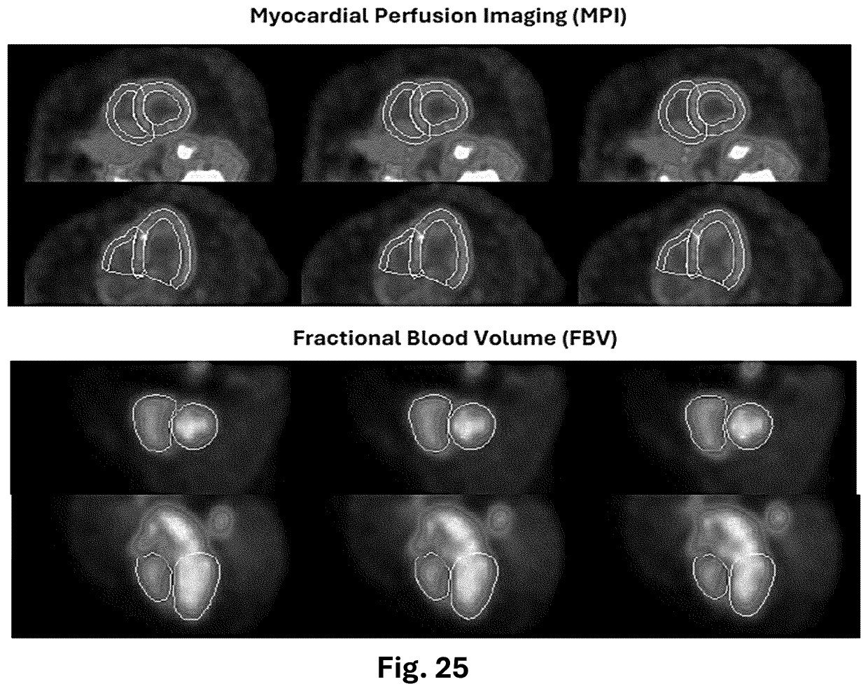

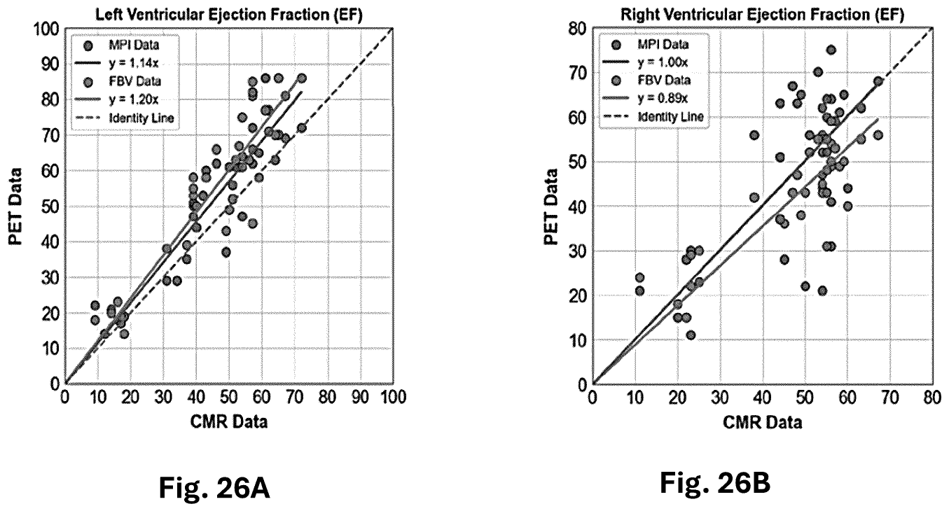

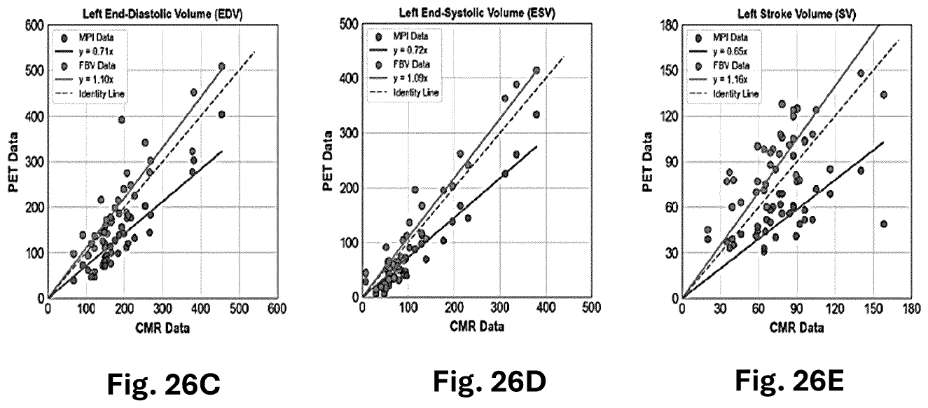

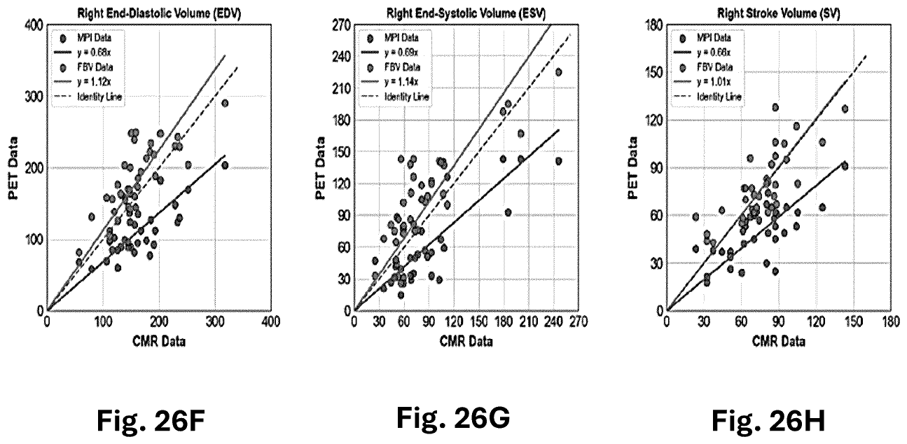

Comparative analysis using CMR as the reference standard demonstrated that the AI-generated BV parametric maps provided clear visualization of both LV and RV blood pools. For the LV, variance (62) was similar between BV and MPI methods. However, MPI overestimated LVEF by 15% and underestimated all volumes by more than −31%, whereas the BV method only overestimated LVEF by 20% and EDV by 25%. For the RV, the BV method yielded significantly lower variance than MPI across all metrics (greater than 31% reduction) and overestimated EDV by 15%. In contrast, MPI overestimated EDV by 34% and underestimated both ESV and EDV by more than −35% (as shown in FIG. 25 and FIGS. 26A-H).

### Conclusion

The inventors of the present invention found that AI-generated gated BV parametric imaging with 82Rb PET reliably estimated volume and ejection fraction of the left and right ventricle. Further, by integrating AI-driven parametric imaging to standard myocardial perfusion and functional assessments in a single modality, 82Rb PET may offer an efficient and comprehensive cardiac function evaluation, extending to the RV.

### Example 7: Multicenter Clinical Validation of an Artificial Intelligence-based Tool for Myocardial Blood Flow Parametric Mapping to Diagnose Coronary Artery Disease with 82Rb PET

### Data Preparation

In an embodiment of the present invention, N=5,614 patients from 16 scanning sites in the US and Canada with 82Rb rest/stress myocardial perfusion imaging (MPI) scans were used as the training set. Two test sets for patients having undergone invasive coronary angiography (ICA) were used for validation: (1) N=1,731 patients from one of the same centers as the training set, and (2) N=1,589 from an external center. Image-derived arterial blood input functions (AIF) and voxel time activity curves (TACs) of the (1) left and (2) right ventricle cavities, (3) voxels with low standard deviation for background TACs, and (4) polar map TACs with were extracted using FlowQuant software. AIFs and TACs were interpolated to equally spaced time frames and normalized by the AIF max.

### Model Training and Evaluation

In the present invention, the AIFs and voxel TACs were fed to a Convolutional/Long-Short Term Memory Neural Network (ConvLSTM) to predict K1, TBV, DV and the associated theoretical TACs according to the one tissue compartment model (1TCM). The AI model was optimized to minimize the mean squared error (MSE) between the observed and theoretical TACS (FIG. 24). For both test sets, global (LV) and regional (LAD, LCx, RCA) rest/stress MBF and reserve (MFR) were extracted from AI-MBF maps and polar processing. The inventors of the present invention computed total perfusion deficit (TPD) and focally impaired myocardial extent from integrated myocardial flow reserve (IMFR) for both AI-MBF and polar processing. Repeated cross-validation logistic regression for predicting CAD (≥70% stenosis) from each biomarker was used to estimate ΔAUC (AI-polar) and the 95% confidence interval (CI) (FIG. 24). Blood input functions and time activity curves (TAC) entered a Convolutional Long-Short Term Memory (ConvLSTM) network to predict K1, TBV, and Distribution Volume (DV=K1/k2). The predicted K1, TBV, and DV entered the 1TCM equation to generate a predicted voxel TAC. The mean squared error (MSE) between the predicted and measured voxel TAC were computed and back-propagated through the network to update model weights. AI-generated MBF maps are projected to polar map space to generate MBF-derived total perfusion deficit (TPD) and focally impaired myocardium extent (iMFR), analogous to conventional polar processing with relative uptake and myocardial flow reserve (MFR). Biomarkers from both methods entered a logistic regression analysis where the area under the curves (AUC) and 95% confidence intervals (CI) were estimated.

### Results

### Parametric Map Agreement

TABLE 2
Areas under the curve (AUC) and 95% confidence intervals (CI) from the logistic
regression clinical validation for AI-MBF vs conventional polar processing.

| | Global | | | Regional (Per-Vessel) | | |
|---|---|---|---|---|---|---|
| Metric | AI AUC | Polar AUC | ΔAUC | AI AUC | Polar AUC | ΔAUC |

External Test Set 1

| | | | | | | |
|---|---|---|---|---|---|---|
| TPD | 0.76 | 0.74 | 0.03* | 0.75 | 0.73 | 0.02* |
| | [0.73; 0.80] | [0.70; 0.77] | [0.01; 0.05] | [0.74; 0.77] | [0.71; 0.75] | [0.01; 0.04] |
| iMFR | 0.77 | 0.75 | 0.02 | 0.75 | 0.75 | 0.00 |
| | [0.74; 0.79] | [0.72; 0.78] | [−0.02; 0.05] | [0.73; 0.77] | [0.73; 0.77] | [−0.01; 0.01] |

External Test Set 2

| | | | | | | |
|---|---|---|---|---|---|---|
| TPD | 0.83 | 0.79 | 0.04* | 0.73 | 0.71 | 0.02* |
| | [0.80; 0.86] | [0.76; 0.82] | [0.02-0.06] | [0.71; 0.75] | [0.69; 0.73] | [0.00; 0.03] |
| iMFR | 0.82 | 0.81 | 0.01 | 0.74 | 0.74 | 0.00 |
| | [0.79; 0.85] | [0.78; 0.84] | [−0.02; 0.04] | [0.72; 0.76] | [0.72; 0.76] | [−0.02; 0.02] |

*p < 0.05

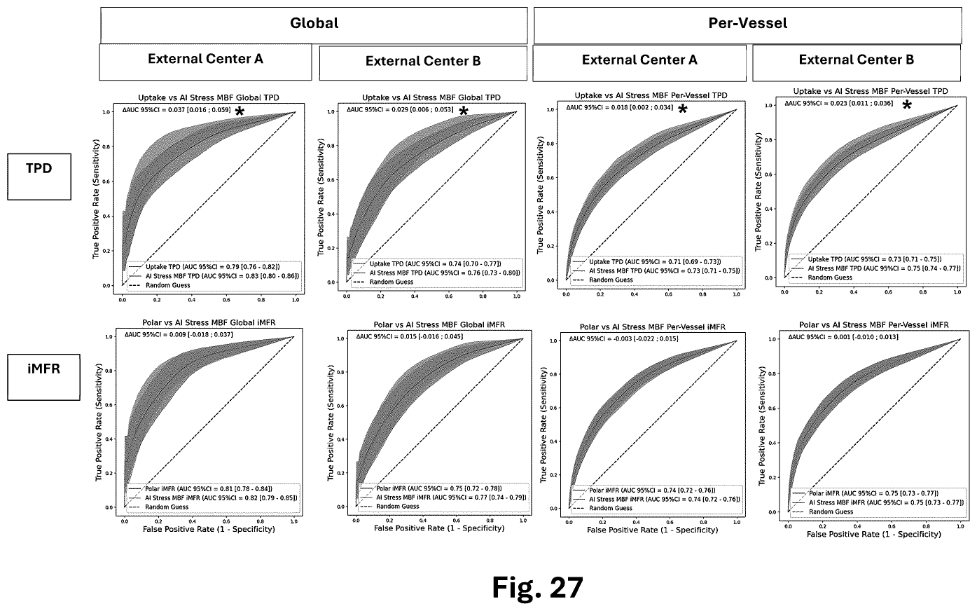

Conclusion: The inventors of the present invention concluded that AI-enabled MBF parametric mapping is more sensitive to relative perfusion defects indicative of CAD and recovers similar absolute flow information as conventional polar map processing, supporting its clinical adoption. In addition, the inventors of the present invention concluded that parametric MBF mapping significantly improved CAD detection for TPD at both the global and regional level and parametric MBF mapping exhibited substantial equivalence for detecting CAD for iMFR focally impaired myocardium extent (as shown in FIG. 27).

What is claimed:

1. A system comprising a method for computing myocardial blood flow (MBF) and related biomarkers using artificial intelligence, the method comprising:
receiving arterial input functions (AIFs) and voxel time activity curves (TACs);
feeding the AIFs and voxel TACs into a convolutional long short-term memory neural network (ConvLSTM);
predicting kinetic parameters including K1, total blood volume (TBV), and distribution volume (DV) using the ConvLSTM;
generating theoretical TACs using a one tissue compartment model (1TCM) based on the predicted parameters; and
optimizing the ConvLSTM model by minimizing the mean squared error (MSE) between observed and theoretical TACs.

2. The system according to claim 1, wherein the distribution volume (DV) is computed as the ratio of K1 to k2.

3. The system according to claim 1, wherein the ConvLSTM model is trained using repeated cross-validation.

4. The system according to claim 1, wherein the theoretical TACs are generated voxel-wise.

5. The system according to claim 1, wherein the mean squared error (MSE) back-propagation includes optimization through the ConvLSTM network.

6. The system according to claim 1, wherein the method further comprises extracting global (LV) and regional MBF and myocardial flow reserve (MFR) from AI-MBF maps and polar processing.

7. The system according to claim 6, wherein global (LV) and regional MBF includes left anterior descending (LAD), left circumflex (LCx), and right coronary artery (RCA) territories, and reverse MFR are extracted for AI-MBF maps and polar processing.

8. A system for diagnosing coronary artery disease (CAD) using AI-derived myocardial biomarkers, comprising:
a ConvLSTM neural network configured to predict K1, TBV, and DV from AIFs and voxel TACs;
a module for generating theoretical TACs using the 1TCM equation:
$${C}_{m} ( t ) = TBV \cdot {C}_{a} ( t ) + RC \cdot {K}_{1} {e}^{{-}^{{K}_{1} / {DV}^{\cdot t}}} ⊗ {C}_{a} ( t ) ;$$
wherein, RC is (1-TBV) for LV polar and background TACs; and RC=1 for LV and RV blood pool TACs;
a polar map projection module for converting AI-generated MBF maps into polar space;
a biomarker extraction module configured to compute total perfusion deficit (TPD) and integrated myocardial flow reserve (iMFR); and
a logistic regression module configured to predict CAD and estimate area under the curve (AUC) and confidence intervals (CI) from the extracted biomarkers.

9. The system according to claim 8, wherein the polar map projection module uses AI-MBF maps to compute TPD and iMFR.

10. The system according to claim 8, wherein the logistic regression module uses biomarkers from both AI-derived and conventional polar processing methods.

11. The system according to claim 8, wherein the logistic regression module estimates AUC and CI for predicting CAD with ≥70% stenosis.

12. The system of claim 8, wherein the ConvLSTM neural network is configured to receive both AIFs and voxel TACs as time-series inputs.

13. The system according to claim 8, wherein the biomarker extraction module computes focally impaired myocardial extent from iMFR.

14. The system according to claim 8, wherein the polar map projection module performs analogous processing to conventional relative uptake methods.

15. A system comprises a method for estimating biventricular cardiac function using 82Rb positron emission tomography (PET), comprising:
acquiring gated dynamic PET imaging data of a subject heart;
reconstructing the gated dynamic PET data into time-resolved images;
extracting arterial input functions (AIFs) from the reconstructed images;
inputting the gated time activity curves and AIFs into a pre-trained convolutional long short-term memory (ConvLSTM) neural network;
generating gated fractional blood volume (FBV) parametric maps from the neural network; and
estimating end-diastolic volume (EDV), end-systolic volume (ESV), stroke volume (SV), and ejection fraction (EF) for both left and right ventricles from the FBV parametric maps.

16. The system according to claim 15, wherein the dynamic PET imaging data is acquired using a list-mode protocol and reconstructed using ordered subset expectation maximization (OSEM) with multiple time frames and ECG-gated bins.

17. The system according to claim 15, wherein the fractional blood volume (FBV) parametric maps are processed using software to derive biventricular functional parameters.

18. The system according to claim 15, wherein the ConvLSTM neural network is trained on gated PET images and corresponding cardiovascular magnetic resonance (CMR) measurements.

19. The system according to claim 15, wherein the fractional blood volume (FBV) parametric maps provide enhanced visualization of right ventricular blood pools compared to conventional gated myocardial perfusion imaging (MPI).

20. The system according to claim 15, wherein the estimated biventricular parameters are validated against CMR-derived measurements using correlation and Bland-Altman analyses.
